(19) United States
(12) Patent Application Publication
(10) Pub. No.: US 2019/0183939 A1
(43) Pub. Date: Jun. 20, 2019

(54) CELL THERAPY USING MIDBRAIN-TYPE NEURAL STEM CELLS TREATED WITH VITAMIN C

(71) Applicant: INDUSTRY-UNIVERSITY COOPERATION FOUNDATION HANYANG UNIVERSITY, SEOUL (KR)

(72) Inventors: Sang-Hun LEE, Seoul (KR); Noviana WULANSARI, Seoul (KR)

(73) Assignee: INDUSTRY-UNIVERSITY COOPERATION FOUNDATION HANYANG UNIVERSITY, SEOUL (KR)

(21) Appl. No.: 16/202,218

(22) Filed: Nov. 28, 2018

(30) Foreign Application Priority Data
Nov. 28, 2017 (KR) 10-2017-0160892

(51) Int. Cl. A61K 35/30; C12N 5/0797; A61P 25/28

(52) U.S. Cl. CPC A61K 35/30; C12N 2500/38; A61P 25/28; C12N 5/0623

(57) ABSTRACT

The present invention relates to a cell therapeutic agent including ventral midbrain-type neural stem cells (VM-NSCs) obtained by treating vitamin C during cell expansion to prevent or treat a neurological disease. Since the vitamin C treatment during NSC expansion prevents the loss of therapeutic function-related NSC characteristics, such as expression of midbrain-specific factors, in the cell expansion, a safe, simple and effective method for mass-producing a cell therapeutic agent with excellent therapeutic functions is provided.

### BACKGROUND

### 1. Field of the Invention

The present invention relates to a cell therapeutic agent containing vitamin C-treated ventral midbrain-type neural stem cells (VM-NSCs).

### 2. Discussion of Related Art

Parkinson's disease (PD) is a common neuro-degenerative disorder characterized by progressive degeneration of dopamine (DA) neurons in the midbrain substantia nigra. Given the well-defined brain region and neuronal type affected, PD is one of the prime target disorders for cell-based therapies. Fetal midbrain transplantation has been clinically performed in PD patients, and has resulted in therapeutic effects. However, limited donor tissue, inconsistent therapeutic outcomes, and dyskinesia side effects prevented this approach from becoming a generalized therapeutic tool. These problems could be solved by developing a standardized donor cell system in which the quality and quantity of transplanted DA neurons could be systematically manipulated. In this regard, culturing neural stem/precursor cells (NSCs) derived from dopaminergic ventral midbrain (VM) tissues is one of the prime candidate cell sources for PD therapy.

In the developing brain, midbrain-type DA (mDA) neurons expressing midbrain-specific markers, such as Foxa2, Lmx1a/b, and Nurr1, arise during early embryonic ventral midbrain (VM) development. Consistent with this, mDA neurons are generated efficiently in vitro in NSCs cultured from the VM during early embryonic days, such as rat embryonic day 11-12 (E11-12). DA neurogenic potential, however, declines severely during in vitro NSCs expansion. In addition, midbrain marker expression is lost in DA neurons differentiated from VM-NSCs during culturing. Of note, expression of midbrain-specific markers is critical for mDA neuron functions, survival, and phenotype maintenance. Additionally, NSCs expanded in vitro exhibit increased apoptotic cell death during/after differentiation, resulting in poor graft formation after transplantation. Methods to halt these culture-dependent changes will need to be developed to generate a systematic source of therapeutically competent donor cells for use in the cell therapeutic approaches for PD.

Vitamin C (L-ascorbic acid; VC) is a crucial micro-constituent in most tissues [Monfort, A., and Wutz, A. (2013). Breathing-in epigenetic change with vitamin C. EMBO reports 14, 337-346]. VC concentration is highest in the brain, and VC assists in multiple functions, including anti-oxidant protection, neurotransmission modulation, myelin formation, and synaptic potentiation [Harrison, F. E., and May, J. M. (2009). Vitamin C function in the brain: vital role of the ascorbate transporter SVCT2. Free radical biology & medicine 46, 719-730]. Brain VC levels are even higher during embryonic development, suggesting specific VC roles during brain development. However, systematic analyses on the practical utility of using VC in cell therapy have not been attempted.

To prepare NSCs as a cell therapeutic agent, it is necessary to culture and expand NSCs.

Therefore, the inventors had confirmed that, in VM-NSC expansion (during the preparation of a cell therapeutic agent), VC treatment provides an effect of reinforcing cell therapy for a neurological disease, and thus the present invention was completed.

### SUMMARY OF THE INVENTION

The present invention is directed to providing a cell therapeutic agent containing vitamin C-treated VM-NSCs.

In addition, the present invention is directed to providing a method of preparing NSCs expressing a midbrain-specific factor, which includes treating vitamin C in the expansion of VM-NSCs.

In addition, the present invention is directed to providing a method of treating a neuronal disease, which includes administering vitamin C-treated VM-NSCs to a subject.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIGS. 1-4. Vitamin C treatment during VM-NSC proliferation rescued the loss of DA neurogenic potential during in vitro expansion.

FIG. 1. Schematic of the experimental design. [NSCs derived from rat embryonic VM tissues at E12 were expanded in vitro with bFGF in the presence or absence (control) of the antioxidants indicated. Cells were passaged every 4 days, as indicated. At each passage, differentiation was induced by removing bFGF from the VM-NSCs expanded, and during the differentiation, VC and the antioxidants were removed, thereby inducing cell differentiation].

FIG. 2. Decline of DA neurogenic potential during in vitro cell expansion with serial passages in control VM-NSC cultures (without antioxidant treatment). [Shown are representative images for TH+DA neurons at differentiation day 6 (D6) in unpassaged cultures (P0, A), and cultures passaged one (P1, B) and two (P2, C) times. Quantitative data of the DA neuronal yields (% TH+ cells of total DAPI+ cells) are depicted as light gray bars in the graph (D). The percentage of TH+ cells in the cultures expanded with VC (200 uM) during P0-P2 are also depicted with dark gray bars in the graph (D). Values represent the mean±SEM; n=3 independent cultures at p<0.05* or p<0.01**, Student's t-test (D), Scale bar, 50 μm].

FIG. 3. Expansion of VM-NSCs for 8 days (up to P1) in the presence or absence of various antioxidants including VC and subsequent induction of differentiation, showing that the loss of yields of differentiated DA neurons in cell expansion is reduced only in a group treated only with VC, and that treatment with an antioxidant other than VC has no such effect [Shown are representative images of TH+DA neurons (A-E) and a quantitative graph (F). Values represent the mean±SEM; n=3 independent cultures at p<0.05* or p<0.01**, and one-way ANOVA with Tukey's post hoc analysis (F). Scale bar, 50 μm].

FIG. 4. VM-NSCs expanded with VC treatment yield TH+DA neurons equipped with other DA neuron phenotypic markers. [P1 cultures expanded with VC were differentiated in the absence of VC for 12 days and subjected to co-immunostaining with TH/DAT (A), TH/AADC (B), and TH/VMAT2 (C). Along with an increase in TH mRNA expression, expression of other DA phenotype markers at D12 increased upon VC treatment during the expansion period (D). *P<0.01, n=3 independent cultures, Student's t-test. Scale bar, 50 μm].

FIG. 5a. Various antioxidant effects during NSC proliferation, related to FIG. 3. Effect of antioxidant treatment on intracellular ROS levels in proliferating VM-NSC cultures assessed by DCF staining in the passaged cultures (P1) at D0. [Shown that, since treatment with an antioxidant other than VC during NSC expansion did not exhibit an effect of enhancing differentiation into DA neurons, the effect of VC treatment on the differentiation into the NSC DA neurons is not related to antioxidation activity of VC].

FIG. 5b. Cell expansion estimated using cell growth curves (B) during VM-NSC passages (P0-P2). [Values represent the mean±SEM; n=3 independent cultures, one-way ANOVA with Tukey's post hoc analysis.]

FIG. 5c. Cell expansion estimated using the population doubling level (PDL) (C) during VM-NSC passages (P0-P2). [Values represent the mean±SEM; n=3 independent cultures, one-way ANOVA with Tukey's post hoc analysis]

FIGS. 6a-6j. VC treatment in VM-NSC expansion and differentiation induced an increase in expression of midbrain-specific factors Nurr1 (FIG. 6a), Foxa2 (FIG. 6b) and Lmx1a (FIG. 6c), known as critical factors for maintaining the survival, functions and characteristics of DA neurons in differentiated DA neurons, as well as an increase in yield of differentiated DA neurons as shown in FIGS. 3 and 4, increases in morphological maturation of DA neurons (FIG. 6d), synaptogenesis (FIG. 6e) and maturation in a functional aspect for secreting dopamine (FIG. 6f), and an increase in cell survival (FIGS. 6g and 6h), and an increase in resistance to various toxins (FIGS. 6i and 6j). VM-NSCs were in vitro expanded with or without (control) VC up to P1, and then differentiated in the absence of VC for 6-15 days. [P<0.05 or **P<0.01, n=3 independent cultures, Student's t-test. Scale bar, 50 μm].

FIGS. 6a-6c. Co-expression of midbrain-specific markers in differentiated DA neurons. Graphs on the right depict the percentage of TH+ cells co-expressing the midbrain-specific markers Nurr1 (FIG. 6a), Foxa2 (FIG. 6b), and Lmx1a (FIG. 6c) out of the total number of TH+ cells at D6. *P<0.001, n=30 microscopic fields, Student's t-test. Arrows in the images indicate TH+DA neurons co-expressing the midbrain markers.

FIG. 6d. Morphological, synaptic, and functional maturity of DA neurons estimated by TH+ fiber length per DA neuron at D15. [n=60 cells].

FIG. 6e. Morphological, synaptic, and functional maturity of DA neurons estimated by number of synapsin+ puncta along the TH+ neurites (100 μm). [n=20 neurites, n=3 independent cultures].

FIG. 6f. Morphological, synaptic, and functional maturity of DA neurons estimated by pre-synaptic DA release. Medium (48 hr), DA levels in the media conditioned in the differentiated cultures for 48 hrs (D13-15); KCl evoked (30 min), DA levels evoked by KCl-mediated depolarization for 30 min. [n=3 independent cultures, *P<0.001, Student's t-test.].

FIGS. 6g-6j. Effect of VC treatment during VM-NSC expansion on differentiated cell survival/death and resistance against toxins. VM-NSCs expanded with or without VC were differentiated for 8 days without VC. General cell death (FIG. 6g) and DNA damage (FIG. 6h) were estimated by the percentage of cells positive for EthD1+ and the percentage of % cells with >3 TH2AX foci at D8, respectively. The differentiated cultures at D8 were exposed to H₂O₂ or 6-OHDA (500 μM and 1000 μM) for 8 hours and viable TH+DA neurons were counted on the following day (FIG. 6i and FIG. 6j). [P<0.05 or **P<0.01, n=3 independent cultures, Student's t-test. Scale bars, 50 μm].

FIGS. 7a-7c. Effect of VC treatment on the expression of general NSC (Nestin, Sox2) and rostral region-specific NSC (Otx2) markers.

FIGS. 7a and 7b. Marker expression was assessed by immunocytochemistry; FIG. 7c. Marker expression was assessed by quantitative real-time PCR. [Values represent the mean±SEM; n=3 independent cultures, t-test.]

FIGS. 8a-8k. VC treatment promoted expression of Foxa2 and Lmx1a in VM-NSCs along with TET1 and Jmjd-mediated changes in global 5hmC/5mC and H3K27m3/H3K9m3 levels.

FIG. 8a. Foxa2 and Lmx1a expression in undifferentiated and differentiated VM-NSC cultures estimated by the percentage of immunoreactive cells among total DAPI+ cells at D0 and D6; FIG. 8b. Foxa2 and Lmx1a expression in undifferentiated and differentiated VM-NSC cultures estimated by real-time PCR at D0. [P<0.05, **P<0.01, n=3 independent cultures, Student's t-test.]

FIG. 8c. Tet enzyme activity in the nuclear fraction at D0. [* P<0.05, n=3 independent cultures (triplicates in each culture), t-test.]

FIG. 8d. Global 5hmC levels estimated by DNA dot blot analyses at D0.

FIG. 8e. Global 5hmC levels estimated by immunocytochemical analyses at D0.

FIG. 8f. Global 5mC levels estimated by DNA dot blot analyses at D0.

FIG. 8g. Global 5mC levels estimated by immunocytochemical analyses at D0.

FIG. 8h. Jmjd3 enzyme activity. [n=3 independent cultures, **P<0.01, t-test].

FIG. 8i. VC effects on global H3K27m3 and H3K9m3 levels estimated by western-blot analyses at D0. [Intensities of the bands were quantified using ImageJ software, and the values normalized to histone 3 (H3) are depicted in the graph on the right. n=3 independent experiments, *P<0.05, t-test.].

FIG. 8j. VC effects on global H3K27m3 level estimated by immunocytochemical analyses at D0. [Scale bars, 50 μm].

FIG. 8k. VC effects on global H3K9m3 level estimated by immunocytochemical analyses at D0. [Scale bars, 50 μm].

FIGS. 9a-9h. VC-induced epigenetic changes on Foxa2 and Lmx1a promoters.

FIG. 9a. Schematics for the rat Foxa2 promoter with CpG enriched regions (indicated with box) targeted for the DIP- and ChIP-PCR analyses.

FIG. 9b. Levels of 5hmC/5mC and H3K27m3/H3K9m3 in the promoter regions of Foxa2 (A and B) in undifferentiated (D0) and differentiated (D6) VM-NSC cultures. [Data are presented as Log 2 values of the fold changes (VC+/VC−). N/A, not amplified. Significance at *P<0.05; **P<0.01; ***P<0.001, n=3 independent experiments, t-test.]

FIG. 9c. Schematics for the rat Lmx1a promoter with CpG enriched regions (indicated with box) targeted for the DIP- and ChIP-PCR analyses. [In the Lmx1a promoter, the CpG region predicted to be a consensus Foxa2 binding (FB) site was analyzed.]

FIG. 9d. Levels of 5hmC/5mC and H3K27m3/H3K9m3 in the promoter regions of Lmx1a (E and F) in undifferentiated (D0) and differentiated (D6) VM-NSC cultures. [Data are presented as Log 2 values of the fold changes (VC+/VC−). N/A, not amplified. Significance at *P<0.05; **P<0.01; ***P<0.001, n=3 independent experiments, t-test.]

FIG. 9e. TET1, Jmjd3, and Jmjd2 protein recruitments to the Foxa2 promoter at D0.

FIG. 9f. TET1, Jmjd3, and Jmjd2 protein recruitments to the Lmx1a promoter at D0.

FIG. 9g and FIG. 9h. VC-induced epigenetic changes on Foxa2 (FIG. 9g) and Lmx1a (FIG. 9h) promoters. [The VC-induced epigenetic changes was abolished by blocking SVCT activity by lowering pH (pH5) and with treatment by the SVCT2 inhibitor; Quercetin (10 μM). The inhibitors and vehicle (-) were treated for 3 hours prior to VC treatment. The levels of the epigenetic proteins and codes were determined in region. Significance at *P<0.05; **P<0.01; n=3 independent experiments, t-test.]

FIGS. 10a and 10b. VC-induced epigenetic changes in later developmental and differentiated mDA neuronal genes. [Undifferentiated VM-NSCs were proliferated in the presence (VC+) or absence of VC (VC−), and then differentiation without VC for 6 days.]

FIG. 10a. Expression of the later mDA developmental gene Nurr1 assessed by immunocytochemical analyses. [Scale bar: 50 μm.]

FIG. 10b. Expression of the later mDA developmental gene Nurr1 assessed by real-time qPCR analyses. [*P<0.05,**P<0.01, ***P<0.001, n=3 independent cultures, Student's t-test,]

FIGS. 10c-10f. Effects of VC treatment on the levels of 5hmC/5mC and H3K27m3/H3K9m3 on the Nurr1 promoter.

FIG. 10c. Schematic for the rat Nurr1 promoter with consensus Foxa2 binding sites (FB) targeted for the DIP- and ChIP-PCR analyses.

FIG. 10d. The hMeDIP/MeDIP-qPCR for 5hmC/5mC (A) and ChIP-qPCR analyses for H3K27m3/H3K9m3 (B) were carried out over the differentiation period (D0-D6).

FIG. 10e shows the increase of Foxa2 protein recruitment to the Nurr1 promoter at D2. [n=3 independent experiments]

FIG. 10f. Along with increases of Foxa2 protein recruitment to the Nurr1 promoter at D2, the percentage of Foxa2+, Nurr1+ cells among total Nurr1+ cells was greater in cultures differentiated from VC-treated NSCs. [n=3 independent cultures, *P<0.001, Student's t-test.].

FIGS. 10g and 10h. Effects of VC treatment on the levels of 5hmC/5mC and H3K27m3/H3K9m3 in the Th gene promoter regions.

FIG. 10g. Rat TH promoter having consensus Nurr1-binding (NB) and 10 CpG sites targeted by DIP- and ChIP-PCR analyses to assess epigenetic code changes.

FIG. 10h. Results of DIP-qPCR(A) analysis for 5hmC/5mC and ChIP-qPCR(B) analysis for H3K27m3/H3K9m3. [performed over the differentiation period (D0 to D6)].

FIG. 10i. Nurr1 protein recruitment to the TH promoter regions at D6. [n=3 independent experiments].

FIG. 10j. Nurr1 efficiency in inducing TH expression estimated by the percentage of Nurr1+, TH+ cells among total Nurr1+ cells at D6. [P<0.05; **P<0.01; ***P<0.001, n=3 independent cultures, Student's t-test].

FIG. 11. SVCT blocker abolished VC-induced epigenetic changes on Nurr1 and Th promoter, related to FIGS. 10a-10j. [VC-induced epigenetic changes on Nurr1 promoter (A) and Th Promoter (B) were abolished by blocking SVCT activity by lowering pH and with treatment of the SVCT2 inhibitor; Quercetin (10 μM). The inhibitor and vehicle (DMSO) were treated for 3 hours prior to VC treatment Significance at *P<0.05; **P<0.01; n=3 independent experiments, t-test].

FIGS. 12a-12e. VC-induced epigenetic changes on the promoters specific for other neuronal subtypes and astrocyte genes, related to FIGS. 10a-10j [VM-NSCs were proliferated in the presence (VC+) or absence of VC (VC−), and differentiated without VC.].

FIG. 12a. The levels of 5hmC/5mC and H3K27m3/H3K9m3 were estimated in the promoter regions (indicated in the schematics for the promoters) of GAD67 (GABAergic neuron, A).

FIG. 12b. The levels of 5hmC/5mC and H3K27m3/H3K9m3 were estimated in the promoter regions (indicated in the schematics for the promoters) of TPH2 (serotonergic neuron.

FIG. 12c. The levels of 5hmC/5mC and H3K27m3/H3K9m3 were estimated in the promoter regions (indicated in the schematics for the promoters) of GFAP (astrocyte).

FIG. 12d. qPCR analyses for GAD67, Tph2, and GFAP mRNA expressions.

FIG. 12e. VC effect on astrocyte differentiation [The VC effect on astrocyte differentiation was further confirmed by GFAP+ astrocyte yields. Proliferating VM-NSCs were passaged in the presence or absence of VC. Six days after differentiation of the cells at each cell passage, GFAP+ cells were counted. Scale bar, 100 μm. Data are presented as the mean±SEM. Significance at *P<0.05; **P<0.01; ***P<0.001, n=3 independent experiments, t-test.].

FIGS. 13a and 13b. VC-treated NSC transplantation into Parkinson's Disease Rat Model. [NSCs derived from rat embryonic VM at E12 were expanded in vitro with or without VC for 8 days. The donor cells were harvested (equivalent to P2 culture), and intrastially transplanted into 6-OHDA-lesioned PD model rats.]

FIG. 13a. Behavioral (A-D) and histological (E-G) analyses were carried out during or at 8 weeks post-transplantation. A-C, Amphetamine-induced rotation scores. A-C, Amphetamine-induced rotation scores. The ipsilateral net rotation values of individual animals transplanted with control-NSCs (A) and VC-NSCs (B) and their mean±SEM (C) are depicted. n=8 rats (VC-NSC), 7 rats (control-NSC). Significant differences from the control at each post-transplantation time point at *P<0.05; **P<0.01, two-way ANOVA. Behaviors of the transplanted animals were further assessed by Stepping test (D) at 8 weeks post-transplantation. *P<0.001, ANOVA followed by Tukey's post-hoc analysis. Histologic analyses 8 weeks post-transplantation. Graft volume (E), Total number of TH+ cells in graft per animal (F), and TH+ cell density in graft (G). *P<0.05; **P<0.01; ***P<0.001, t-test].

FIG. 13b. (A, B): Overviews of TH+ cell grafts. [Insets, TH+DA neuron morphology in grafts shown with high magnification. Scale bars, 500 μm].

FIG. 13b. (C-K): Co-expression of mature neuronal (HuC/D and NeuN), midbrain-specific (Foxa2 and Nurr1), and A9 nigral mDA neuronal (Girk2) markers in TH+DA cells at 8 weeks post-transplantation. (L): DARPP-32+ striatal neurons adjacent to TH+ cells in the VC-NSC graft. [Scale bars, 30 μm].

FIGS. 14a-14f. Histological analysis at 2 months after transplantation, related to FIGS. 13a and 13b.

FIG. 14a. Representative images of cleaved (activated) caspase-3+ cells in graft. [Right: Graph depicting cleaved caspase-3+ cells/mm³ in graft. Values represent mean±SEM; n=3 animals; *P<0.05, t-test. Scale bar, 30 μm].

FIG. 14b. Representative images of the proliferating cell-specific nuclear antigen (PCNA)+ cell in graft.; FIG. 14c. Representative images of the phospho-histone H3 (pHH3, mitosis marker)+ cell in graft. [It is noted that none are positive for the proliferating cell-specific markers in both the control VC- and VC+ grafts, Values represent mean±SEM; n=3 animals; *P<0.05, t-test. Scale bar, 30 μm].

FIG. 14d. Images of HuC/D+ cells in graft. [Right: Graph depicting of HuC/D+ cells/mm³ in graft, Values represent mean±SEM; n=5 animals; *P<0.05, t-test. Scale bar, 30 μm].

FIG. 14e. Representative images of 5-HT+ cells in graft. [Right: Graph depicting 5-HT+ cells/mm³ in graft. Values represent mean±SEM; n=3 animals; *P<0.05, t-test. Scale bar, 30 μm].

FIG. 14f. Representative images of TH+, GFAP+ cells in graft. [Right: Graph depicting GFAP+ cells/mm³ in graft. Values represent mean±SEM; n=5 animals; *P<0.05, t-test. Scale bar, 30 μm].

FIGS. 15a-15e. Effects of VC treatment in human NSC cultures derived from hESCs, related to FIGS. 6a-6j and 10a-10j. [The human NSCs derived from hESCs (H9) were proliferated in the presence (VC+) or absence of VC (VC−), and then differentiation was induced without VC for 6 days.]

FIG. 15a. Effect of VC treatment on differentiated cell survival/death. [General cell deaths were estimated by the percentage of cells positive for EthD1 at differentiation day 6 (D6), n=3 independent cultures. Scale bar, 50 μm.]

FIG. 15b. % TH+DA neurons at D6. [Scale bar, 50 μm. n=3 independent cultures].

FIG. 15c. Morphological maturation of the differentiated DA neurons estimated by TH+ fiber lengths per DA neuron at D15, n=50 cells.

FIG. 15d. qRT-PCR analysis exhibiting VC effect on human Th genes expression. [n=3 independent cultures.]

FIG. 15e. 5hmC/5mC levels on the human Th promoter (Consensus NBRE regions). [DIP-qPCR were carried out at D6. n=3 independent experiments. Values represent mean±SEM. Significantly difference from untreated control (VC−) at *P<0.05; **P<0.01; t-test.]

### DETAILED DESCRIPTION OF EXEMPLARY EMBODIMENTS

Hereinafter, the present invention will be described with reference to examples and comparative examples in detail. However, the present invention is not limited to these examples.

The present invention relates to a cell therapeutic agent containing vitamin C-treated VM-NSCs.

The term “neural stem cells (NSCs)” used herein refers to all of neural stem cells that express or do not express a midbrain-specific factor.

In one exemplary embodiment of the present invention, the term “ventral midbrain-type NSCs (VM-NSCs)” may be the NSCs expressing a midbrain-specific factor, which is selected from the group consisting of Foxa2, Lmx1a and Nurr1.

In one exemplary embodiment of the present invention, the vitamin C-treated NSCs are prepared by treating vitamin C in NSC expansion.

The “treatment” used herein refers to all actions involved in preventing, alleviating or beneficially changing clinical situations related to a disease. In addition, the treatment may refer to an increased survival compared with an expected survival rate when untreated. The treatment includes a preventive means in addition to a therapeutic means.

The term “vitamin C treatment” used herein refers to treatment of vitamin C in NSC expansion before transplantation of midbrain-type NSCs, and thus means that vitamin C is not treated after transplantation or during in vitro differentiation.

The term “cell therapeutic agent” used herein refers to a medicine for administering a genetic material or cells containing a genetic material to a human body in order to treat a disease or the like.

The cell therapeutic agent of the present invention may be administered in various routes including oral and non-oral routes to reach desired tissues. The cell therapeutic agent of the present invention may be administered orally or non-orally, for example, intraperitoneally, intravenously, intramuscularly, subcutaneously, intradermally, locally, intranasally, intrapulmonarily, or rectally, but the present invention is not limited thereto.

The cell therapeutic agent may be formulated in a suitable form with a pharmaceutically acceptable carrier generally used in the art. The term “pharmaceutically acceptable carrier” refers to a carrier or excipient useful in preparing a composition that is physiologically acceptable, and does not generally cause an allergic reaction such as a gastrointestinal disorder or dizziness, or a similar reaction thereto when administered to a human. The pharmaceutically acceptable carrier may include a carrier for non-oral administration, for example, water, suitable oil, saline, aqueous glucose or glycol, and further include a stabilizer and a preservative. As a suitable stabilizer, an antioxidant such as sodium hydrogen sulfite, sodium sulfite or ascorbic acid may be used. A suitable preservative may be benzalkonium chloride, methyl- or propyl-paraben, or chlorobutanol. Other pharmaceutically acceptable carriers may be selected by referring to the following literature (Remington's Pharmaceutical Sciences, 19th ed., Mack Publishing Company, Easton, Pa., 1995).

A composition for the cell therapeutic agent according to the present invention may include a therapeutically effective amount of the cell therapeutic agent to treat a disease.

The term “therapeutically effective amount” refers to an amount of an active ingredient or pharmaceutical composition considered by a researcher, a veterinarian, a doctor or a clinical trial as being capable of inducing a biological or medical reaction in a tissue system, an animal or a human, and includes an amount of inducing alleviation of the symptoms of a disease or disorder to be treated. It is apparent to those of ordinary skill in the art that the cell therapeutic agent included in the composition of the present invention may be varied according to a desired effect. Therefore, an optimal content of the cell therapeutic agent may be easily determined by one of ordinary skill in the art, and may be adjusted according to various factors such as the type and severity of a disease, contents of other components in the composition, the type of a dosage form, and a patient's age, body weight, general health condition, sex and diet, administration time, an administration route, a secretion rate of the composition, the duration of treatment, and a concurrently-used drug. It is important to include an amount that can achieve the maximum effect with the minimum amount without a side effect in consideration of all of the above factors. For example, the composition of the present invention may include the cell therapeutic agent at 1×10⁴ to 1×10⁸ cell/kg.

The present invention provides a use of the composition including a cell therapeutic agent for preparing a medicine for preventing or treating a neurological disease as an active ingredient. The composition of the present invention including the cell therapeutic agent as an active ingredient may be used to prepare a drug for preventing or treating a neurological disease.

The present invention also provides a pharmaceutical composition for preventing or treating a neurological disease, which includes vitamin C-treated VM-NSCs.

The neurological disease may be selected from the group consisting of Parkinson's disease, Alzheimer's disease, Huntington's disease, amyotrophic lateral sclerosis, stroke, ischemia and neurological diseases caused by spinal cord injury, but the present invention is not limited thereto.

The present invention also provides a method of treating a neurological disease, which includes administering vitamin C-treated VM-NSCs to a subject.

In this specification, the term “subject” may refer to a vertebrate to be tested for treatment, observation or experiments, preferably a mammal, for example, a dog, a cat, a rat, a human, etc.

In the therapeutic method of the present invention, for an adult, the composition for a cell therapeutic agent according to the present invention may be administered one to several times a day, and preferably includes the cell therapeutic agent at 1×10⁴ to 1×10⁸ cell/kg.

In the therapeutic method of the present invention, the composition including the cell therapeutic agent of the present invention as an active ingredient may be administered by a conventional route, for example, rectally, intravenously, intraarterially, intraperitoneally, intramuscularly, intrasternally, subcutaneously, locally, intraocularly or transdermally.

In addition, the composition according to the present invention includes a pharmaceutically acceptable carrier, in addition to the stem cells as an active ingredient. The term “pharmaceutically acceptable carrier” refers to a carrier or excipient useful in preparing a composition that is physiologically acceptable, and does not generally cause an allergic reaction such as a gastrointestinal disorder or dizziness, or a similar reaction thereto when administered to a human. The pharmaceutically acceptable carrier may include a carrier for oral administration such as lactose, starch, a cellulose derivative, magnesium stearate or stearic acid, and a carrier for non-oral administration such as water, suitable oil, saline, aqueous glucose or glycol, and further include a stabilizer and a preservative. As a suitable stabilizer, an antioxidant such as sodium hydrogen sulfite, sodium sulfite or ascorbic acid may be used. A suitable preservative may be benzalkonium chloride, methyl- or propyl-paraben, or chlorobutanol. Other pharmaceutically acceptable carriers may be selected by referring to the following literature (Remington's Pharmaceutical Sciences, 19th ed., Mack Publishing Company, Easton, Pa., 1995). The pharmaceutical composition according to the present invention may be formulated in a suitable form according to a method known in the art, together with the above-described pharmaceutically acceptable carrier. In other words, the pharmaceutical composition of the present invention may be prepared in various forms for non-oral administration according to a known method, and the representative form for non-oral administration is preferably an isotonic aqueous solution or a suspension. An injectable form may be prepared with a suitable dispersing agent or wetting agent, and a suitable dispersing agent according to technology known in the art. For example, an injectable form may be prepared by dissolving each component in saline or buffer.

An effective amount of the pharmaceutical composition formulated by the above-described method may be administered in various routes, for example, by transdermal, subcutaneous, intravenous or intramuscular administration. The “effective amount” used herein refers to an amount capable of exhibiting a preventive or therapeutic effect when administered to a patient. The effective amount of the pharmaceutical composition according to the present invention may be suitably selected according to an administration route, an administration target, age, sex, a body weight, personal characteristics and a diseased state. A content of the active ingredient in the pharmaceutical composition of the present invention may be varied according to a severity of the disease, and the active ingredient is administered several times daily at an effective content of, preferably, 1-10000 μg/kg of body weight/day, and more preferably, 10-1000 mg/kg of body weight/day. In addition, the composition for a cell therapeutic agent for treating a neurological disease according to the present invention may be administered in combination with a known compound having an effect of preventing, improving or treating a neurological disease.

In addition, the present invention includes

a method of preparing NSCs expressing a midbrain-specific factor, which includes:

treating vitamin C in VM-NSCs expansion.

The vitamin C is preferably treated at 50 to 300 μM. Here, when the vitamin C is treated at less than 50 μM, a vitamin C treatment effect is deteriorated, and when the vitamin C is treated at more than 300 μM, cytotoxicity is induced.

In the method of preparing NSCs, all of the above descriptions may be applied or applied mutatis mutandis to NSCs.

Hereinafter, the present invention will be described in detail with reference to examples thereof. However, it should be understood that the following examples are just preferred examples for the purpose of illustration only and is not intended to limit or define the scope of the invention. The following examples described herein are provided in order to make the present invention more comprehensive and complete and provide the scope of the present invention to those skilled in the art to which the present invention belongs and thus will be defined by the appended claims equivalents thereof.

### EXAMPLES

### 1. Experimental Procedures

Cell Cultures and Chemicals

NSCs were cultured from rat embryo VMs (Sprague Dawley) at embryonic day 12 (E12) on 6-cm dishes or 24-well plates pre-coated with 15 μg/ml poly-L-ornithine (PLO; Sigma)/1 ug/ml fibronectin (FN; Sigma, St. Louis, Mo.) in serum-free N2 medium. NSCs were induced to proliferate by the mitogenic action of basic fibroblast growth factor (bFGF, 20 ng/ml, R&D Systems, Minneapolis, Minn.). The proliferating VM-NSCs cultured in 6-cm dishes were passaged at every 4th day in mitogen-supplemented medium with or without Vitamin C (VC; Sigma; 200 μM), glutathione reduced form (GSH; Sigma; 200 μM), vitamin E (alpha-tocopherol; Sigma; 200 μM), or N-acetylcysteine (NAc; Sigma; 100 μM). The expanded NSCs at the last day of each passage were induced to differentiate by withdrawing the mitogen and antioxidants from the media (for 2-16 days). Human NSC cultures were derived by in vitro differentiation of hESCs (H9) and cultured as described in the following reference. [Rhee, Y. H., Ko, J. Y., Chang, M. Y., Yi, S. H., Kim, D., Kim, C. H., Shim, J. W., Jo, A. Y., Kim, B. W., Lee, H., et al. (2011). Protein-based human iPS cells efficiently generate functional dopamine neurons and can treat a rat model of Parkinson disease. The Journal of clinical investigation 121, 2326-2335]. The human NSCs were expanded in ITS media supplemented with bFGF (20 ng/ml) in the presence or absence of VC for two NSC passages. Differentiation of the passaged human NSCs was induced by withdrawal of bFGF and VC in ITS supplemented with brain-derived neurotrophic factor (20 ng/ml; R&D Systems), glial cell line derived neurotrophic factor (20 ng/ml; R&D Systems), and dibutyryl cAMP (0.5 mmol/1; Sigma). Cultures were maintained at 37° C. in humidified 5% CO₂ incubators.

Immunofluorescence Staining

Cultured cells and cryosectioned brain slices were fixed with 4% paraformaldehyde (PFA), and blocked for 40 minutes in blocking solution (Blocking solution: 1% BSA+0.3% Triton X-100). For 5hmC and 5mC staining, the fixed cells were incubated with 2 N HCl for 20 minutes before the blocking reaction. The samples were incubated overnight at 4° C. in the blocking solution containing the primary antibodies listed in Supplemental Table 1. Secondary antibodies tagged with Alexa 488 (1:200, Invitrogen, Carlsbad, Calif.) and Cy3 (1:200, Jackson Immunoresearch Laboratories, West Grove, Pa.) were applied. Stained samples were mounted in Vectashield medium containing 40,6-diamidino-2-phenylindole (DAPI; Vector Laboratories, West Grove, Pa.) and analysed under an epifluorescence (Leica, Wetzlar, Germany) or confocal microscope (Leica TSP SP5).

TABLE 1
Antibodies Information

| | | |
|---|---|---|
| Name | Company (Cat. No) | Dilution |

Antibodies used in Immunocytochemistry and Immunohistochemistry

| | | |
|---|---|---|
| TH (Rabbit) | Pel-Freez (P40101-150) | 1:1000 |
| TH (Mouse) | Sigma (T1299-2ML) | 1:1000 |
| TH (Mouse) | Immunostar (22941) | 1:200 |
| Nurr1 (Mouse) | Perseus Proteomics (PP-N1404-00) | 1:500 |
| Foxa2 (Goat) | Santa Cruz (M-20) | 1:500 |
| Lmx1a (Rabbit) | Millipore (AB10533) | 1:500 |
| DAT (Rat) | Abcam (ab5990) | 1:200 |
| VMAT2 (Rabbit) | Millipore (AB1598P) | 1:200 |
| AADC (Rabbit) | Protos (CA-201) | 1:200 |
| H2AX (Mouse) | Millipore (05-636) | 1:500 |
| 5hmC (Rabbit) | Activ Motif (39769) | 1:1000 |
| 5mC (Mouse) | Abcam (ab10805) | 1:1000 |
| H3K27m3 (Rabbit) | Millipore (07-442) | 1:1000 |
| H3K9m3 (Rabbit) | Millipore (07-449) | 1:1000 |
| HuCD (Mouse) | Millipore (MABN153) | 1:100 |
| NeuN (Mouse) | Millipore (MAB377) | 1:200 |
| GFAP (Mouse) | MP Biomedicals (08691102) | 1:200 |
| GIRK2 (Rabbit) | Alomone labs (APC-006) | 1:80 |
| DARPP32 (Rat) | R&D System (MAB4230) | 1:80 |
| Nestin (Mouse) | BD Pharmingen (556309) | 1:500 |
| OTX2 (Rabbit) | Millipore (AB 9566-1) | 1:400 |
| SOX2 (Rabbit) | Millipore (AB5603) | 1:500 |
| Synapsin I | BD Biosciences (MAb) | 1:200 |
| PHH3 (Rabbit) | Millipore (06-570) | 1:500 |
| PCNA (Mouse) | Upstate (05-347) | 1:40 |
| 5-HT (Rabbit) | Sigma (S5545) | 1:2000 |
| Cleaved Caspase 3 (Rabbit) | Cell Signalling (D175) | 1:1000 |

Antibodies for ChIP and DIP Analysis

| | | |
|---|---|---|
| 5hmC (Rabbit) | Activ Motif (39769) | 3 μg |
| 5mC (Mouse) | Abcam (ab10805) | 3 μg |
| H3K27m3 (Rabbit) | Millipore (07-442) | 3 μg |
| H3K9m3 (Rabbit) | Millipore (07-449) | 3 μg |
| Nurr1 (Rabbit) | Santa Cruz (N-20) | 3 μg |
| Foxa2 (Rabbit) | Abcam (ab83517) | 3 μg |
| Jmjd3 (Rabbit) | Abcam (ab85392) | 3 μg |
| Jmjd2 (Mouse) | Santa Cruz (D-4) | 2.5 μg |
| TET1 (Rabbit) | Millipore (09-872) | 3 μg |

Cell Growth and Survival Assays

The cell growth profile during NSC expansion with several cell passages was generated by counting the number of viable cells at the end of each passage. Cell expansion of each NSC passage was further estimated by the population doubling level (PDL, the level at which the number of cells is doubled), which was determined by log(N/NO)/log 2 (where N is the number of cells at the end of each passage; NO is the number of cells plated initially (1×10⁵ cells/cm²)). DA neuronal cell survival in the absence or presence of toxic insults (500-1000 uM of H₂O₂ or 6-OHDA) was determined by counting viable TH⁺ cells after staining. General cell death and DNA damage were estimated by ethidium heterodimer 1 (EthD1) (Molecular Probes) and γH2AX staining, respectively.

Morphologic and Functional Maturation of DA Neurons

To estimate morphological maturation, total fiber lengths emanating from TH+DA neuronal cells were measured. The pre-synaptic activity of DA neurons was determined by measuring the levels of DA neurotransmitter released in the differentiated VM-NSC cultures. Media incubated for 2 days (differentiation day 13-15) were collected and used in the DA level determinations using an ELISA kit (BA E-5300, LDN). In addition, DA release evoked by membrane depolarization was estimated by incubating the cultures (at differentiation day 15) in fresh N₂ media in the presence or absence of 56 mM KCl for 30 min. The evoked DA release was calculated by subtracting the DA release without KCl from the DA level with KCl.

Enzyme Activity Assays

TET and JMJD3 activities were measured using Epigenase™ 5mC hydroxylase TET Activity/Inhibition Assay Kit and Epigenase™ JMJD3/UTX Demethylase Activity/Inhibition Assay Kit (Epigentek, Farmingdale, N.Y.), respectively. Briefly, cells from two 10 cm-dishes were harvested after 4 days of proliferation with or without VC (200 μM) treatment. Nuclear fractions were obtained from the cells using the EpiQuik nuclear extraction kit (Epigentek), and subjected to enzyme activity analyses.

Global Epigenetic Code Determination

DNA dot blot analysis for 5hmC/5mC was performed as described in the following reference. [He, X. B., Kim, M., Kim, S. Y., Yi, S. H., Rhee, Y. H., Kim, T., Lee, E. H., Park, C. H., Dixit, S., Harrison, F. E., et al. (2015). Vitamin C facilitates dopamine neuron differentiation in fetal midbrain through TET1- and JMJD3-dependent epigenetic control manner. Stem Cells 33, 1320-1332]. Briefly, genomic DNA was extracted and quantified. Genomic DNA (80 ng) was spotted on nitrocellulose membranes, air dried, and exposed in UV for 20 minutes. After blocking with 5% bovine serum albumin (BSA)/Tris-buffered saline-Tween 20 at room temperature for 2 hours, the membrane was incubated in anti-5hmC (Active Motif, Carlsbad, Calif.) and anti-5mC (Abcam, Cambridge, UK) antibodies at 4° C. overnight. Western-blots for determining global histone modification changes were performed as described in the following reference. [Rumbaugh, G., and Miller, C. A. (2011). Epigenetic changes in the brain: measuring global histone modifications. Methods Mol Biol 670, 263-274]. Briefly, histones were acid-extracted from cell samples. Histone samples (1m) were electrophoresed on a 15% SDS-PAGE gel, and the blotted membranes were incubated with anti-H3K4m3, H3K9m3, H3K27m3, H3K36m3, and H3 antibodies (all from Millipore, Billerica, Mass.). Positive bands were detected and captured by ChemiDoc (Bio-Rad, Hercules, Calif.), and the intensities of the bands were quantified using ImageJ software (http://imagej.nih.gov/ij).

Chromatin Immunoprecipitation-Quantitative PCR (ChIP-qPCR) and DNA Immunoprecipitation-Quantitative PCR (DIP-qPCR)

Foxa2 and Nurr1 binding sites were identified using the Jaspar database (http://jaspar.genereg.net/) using an 80% score threshold. Predicted Foxa2 and Nurr1 binding sites were coupled with phylogenetic footprinting to eliminate spurious predictions with specified position weight matrix (PWM) settings, as described in the following reference. [Sandelin, A., and Wasserman, W. W. (2004). Constrained binding site diversity within families of transcription factors enhances pattern discovery bioinformatics. Journal of molecular biology 338, 207-215] (Table 2). ChIP and DIP assays were carried out as described in the following reference. [Prolonged membrane depolarization enhances midbrain dopamine neuron differentiation via epigenetic histone modifications. Stem Cells 29, 1861-1873]. Briefly, chromatin or genomic DNA was sheared to an average of 200-500 bp in length using a sonication Bioruptor (BMS, Seoul, Korea) and immunoprecipitated using the antibodies listed in Table 1. Immunoprecipitated DNA fragments were collected using magnetic beads (Invitrogen), purified, and subjected to real-time PCR using the primers listed in Table 3. The comparative cycle threshold method was used to quantify the results. Data were normalized to the input DNA.

TABLE 2
Prediction of Transcription Factor Binding
Site on Gene Promoters

| | | | Prediction | |
|---|---|---|---|---|
| | | | Binding Sites | |
| | Gene | Score | Predicted | |
| TF | Promoter | Threshold | Sequence | Strand |
| Foxa2 | Lmx1a | 80% | GAAAGTTGATTT | + |
| | | | (SEQ ID NO: 1) | |
| | Nurr1 | 80% | CTCTATTGTTTT | + |
| | | | (SEQ ID NO: 2) | |
| | | | TATTATTTATAT | − |
| | | | (SEQ ID NO: 3) | |
| | | | TAATATTTCCTT | + |
| | | | (SEQ ID NO: 4) | |
| Nurr1 | Th | 80% | AAGGTTAA | + |
| | | | AAGGTCAC | + |
| | | | CTGGCCTT | + |
| | | | GAGGTCAG | + |
| STAT3 | GFAP | 85% | TTCCGAGAAG | + |
| | | | (SEQ ID NO: 5) | |

TABLE 3
PCR Primers used in this experiment
Gene Expression Analysis

| Gene | Forward | Reverse |
|---|---|---|
| Gapdh | CTCATGACCACAGTCCATGC | TTCAGCTCTGGGATGACCTT |
| | (SEQ ID NO: 6) | (SEQ ID NO: 7) |
| Th | AAGGGCCTCTATGCTACCCAA | TGCATTGAAACACGCGGAAG |
| | (SEQ ID NO: 8) | (SEQ ID NO: 9) |
| Dat | GGACCAATGTTCTTCAGTGGTGGC | GGGCCGGCGAGGGGCTTGAC |
| | (SEQ ID NO: 10) | (SEQ ID NO: 11) |
| Vmat2 | GGCTTCCTTCTCAACTCCCC | GGCTCTGACGTCACGGATAG |
| | (SEQ ID NO: 12) | (SEQ ID NO: 13) |
| Aadc | TGGCAATTTAAAGGCTCTGGAC | CAGAAAAAGGCTGTCCTGGG |
| | (SEQ ID NO: 14) | G |
| | | (SEQ ID NO: 15) |
| Foxa2 | TATGTGGGCGCTGGAATGAG | GGCACCTTGAGAAAGTCGTTG |
| | (SEQ ID NO: 16) | (SEQ ID NO: 17) |
| Lmx1a | TATACAACGTTGCCCACCCC | GATGGGGTTTCCCACTCTGG |
| | (SEQ ID NO: 18) | (SEQ ID NO: 19) |
| Nurr1 | CGGTTTCAGAAGTGCCTAGC | TTGCCTGGAACCTGGAATAG |
| | (SEQ ID NO: 20) | (SEQ ID NO: 21) |
| Human | TCGCTTCGGCAGCACATATAC | TGCGTGTCATCCTTGCGCAG |
| U6 | (SEQ ID NO: 22) | (SEQ ID NO: 23) |
| snRNA | | |
| Human | GAGTACACCGCCGAGGAGATT | GCGGATATACTGGGTGCACTG |
| Th | (SEQ ID NO: 24) | G |
| | | (SEQ ID NO: 25) |

ChIP-qPCR and DIP-qPCR Analysis

| Region | | Forward | Reverse |
|---|---|---|---|
| Foxa2 | I | CCACCTACTGCCCTGTTTGT | CTCAGGCCTTTTTCTGGCTA |
| Promoter | | (SEQ ID NO: 26) | (SEQ ID NO: 27) |
| | II | TCTCTGGGTTCCCTGTGTTC | CACTTGGGTCTGCATCCTTT |
| | | (SEQ ID NO: 28) | (SEQ ID NO: 29) |
| | III | AGAGGGACGGGGGAATAGAC | GTACTGGACTCCCGAGATGC |
| | | (SEQ ID NO: 30) | (SEQ ID NO: 31) |
| Lmx1a | FB | AGCTCGCCCATCAGGTAAG | GAACGTCTGCAGGCGCAAAG |
| Promoter | | (SEQ ID NO: 32) | (SEQ ID NO: 33) |
| Nurr1 | FB1 | CCACCCTCGCACCCAAATTA | AAACACAGCATCAAAGCCGC |
| Promoter | | (SEQ ID NO: 34) | (SEQ ID NO: 35) |
| | FB2 | AGCATTGTGGAGAAGGTGC | GGGCAAGTGAATTGGTGTTC |
| | | TAA | (SEQ ID NO: 37) |
| | | (SEQ ID NO: 36) | |
| Th | NB1 | AGAGGATGCGCAGGAGGTAG | GTCCCGAGTTCTGTCTCCAC |
| Promoter | | (SEQ ID NO: 38) | (SEQ ID NO: 39) |
| | NB2 | TCCTGGAGGGGACTTTATGA | CTGGATTTCCTAAGGGCTCA |
| | | (SEQ ID NO: 40) | (SEQ ID NO: 41) |
| | NB3 | GGGTGTGGATGCTAACTGGA | AGTGGTAGCCCCATTCTCAG |
| | | (SEQ ID NO: 42) | (SEQ ID NO: 43) |
| | 10 CpG | TCTCAGAGCAGGATGCCAAC | ACAAGATGGGACCAAGAACC |
| | | (SEQ ID NO: 44) | (SEQ ID NO: 45) |
| Gfap | STAT3 | TGACTCACCTTGGCATAGACA | CAGTGAGGCATACGGCAAG |
| Promoter | Binding | (SEQ ID NO: 46) | (SEQ ID NO: 47) |
| Human | NRBE1 | GGGTTGTCCTCAAGGGAGTT | CTATGGCCCACTGCTAGCTC |
| Th | | (SEQ ID NO: 48) | (SEQ ID N0:49) |
| Promoter | NRBE2 | TCCCTTTGCTTTGACTGAGC | GACTTCTCGAAGGCCTACCC |
| | | (SEQ ID NO: 50) | (SEQ ID NO: 51) |
| Tph2 | | CAAAGGGCTACTCGACCTAT | GTGCTGAAGAGCAGTGCGCT |
| Promoter | | (SEQ ID NO: 52) | (SEQ ID NO: 53) |
| Gad67 | | AGACACCTGCAAAGGAGCCC | ACAGGGAGCTGGACCCTCCC |
| Promoter | | (SEQ ID NO: 54) | (SEQ ID NO: 55) |

Transplantation and Histological Procedures

All procedures for animal experiments were approved by the Institutional Animal Care and Use Committee (IACUC) at Hanyang College of Medicine (approval number 2014-0212A). Experiments were performed in accordance with National Institutes of Health (NIH) guidelines. Hemi-parkinsonian was induced in adult female Sprague-Dawley rats (220-250 g) by unilateral stereotactic injection of 3 μl of 6-hydroxydopamine (6-OHDA, 8 μg/μl; Sigma) into the right side of the substantia nigra (AP-4.8 mm, ML 1.5 mm, V 8.2 mm) and the median forebrain bundle (AP-1.8 mm, ML 1.8 mm, V 8.0 mm). The coordinates of Bregma were established by setting the incisor bar at −3.5 mm, and fixing a rat head using the ear bar. An amphetamine-induced rotation test was carried out to determine whether the Parkinson's disease was induced, and rats with 300 turns/hr were selected as Parkinson's disease models. For transplantation, rat E12 VM-NSCs were expanded with or without vitamin C treatment for 8 days (including one passage at day 4), and 3 ul of the single cell dissociates (1.5×10⁵ cells/ul) were injected over a 10 min period into each of two sites in the striatum (coordinates in AP, ML, and DV relative to bregma and dura: (1) 0.07, −0.30, −0.55; (2)−0.10, −0.40, −0.50; incisor bar set at 3.5 mm below zero) under anesthesia induced by Zoletil 100 ul/100 g (50 mg/ml) mixed with Rompun 100 ul/100 g (23.32 mg/ml). The needle (22 gauge) was left in place for 5 min after the completion of each injection. Rats received daily injections of cyclosporine A (10 mg/kg, i.p.) starting 1 day before the grafting and continuing for 1 month thereafter, followed by a reduced dose (5 mg/kg) for the remaining time. Eight weeks after transplantation, animals were anesthetized and perfused transcardially with 4% paraformaldehyde. Brains were removed and immersed in 30% sucrose in PBS overnight, frozen in Tissue-Tek® (Sakura Finetek USA), and then sliced using cryostat (Leica, CM1850). Free-floating brain sections (30 μm thick) were subjected to immunohistochemistry as described above and images were obtained with a confocal microscope (Leica TSP SP5). The graft volume was measured as described in the following reference. [Antidepressants increase neural progenitor cells in the human hippocampus. Neuropsychopharmacology: official publication of the American College of Neuropsychopharmacology 34, 2376-2389, Direct comparison of autologous and allogeneic transplantation of iPSC-derived neural cells in the brain of a non-human primate. Stem cell reports 1, 283-292, Protein-based human iPS cells efficiently generate functional dopamine neurons and can treat a rat model of Parkinson disease. The Journal of clinical investigation 121, 2326-2335, Effect of Rho Kinase Inhibitors on Grafts of Dopaminergic Cell Precursors in a Rat Model of Parkinson's Disease. Stem cells translational medicine 5, 804-815]. Briefly, grafts were clearly identified by staining with TH in the absence of a TH⁺ background in the striatal sections where dopaminergic neuron fibers are denervated in the PD animal model. TH+ grafts from every 5th section were outlined in digitized images and the graft area were calculated using an LAS image analyzer. Graft volume was calculated using Calvalieri's method with the following formula 1:

where T is the distance between parallel sections (in this study, the value was 0.15 mm=30 μm [section thickness]×5), A is the calculated area of a section, and n is the total number of sections.

V(graft volume)=T*Σ_{(i=1→n)}A [formula 1]

Behavior Tests

Animal behaviors were assessed using amphetamine-induced rotation and step adjustment tests, as described in the following reference. [Protein-based human iPS cells efficiently generate functional dopamine neurons and can treat a rat model of Parkinson disease. The Journal of clinical investigation 121, 2326-2335].

Cell Counting and Statistical Analysis

Immunostained cells were counted in 10-20 random areas of each culture coverslip using an eyepiece grid at a magnification of 200×. Data are expressed as the mean±SEM of three to eight independent cultures. For every figure, statistical tests are justified as appropriate. Statistical comparisons were made using Student's t-test (unpaired) or one-way ANOVA followed by Tukey's post hoc analysis using SPSS® Statistics 21; IBM Inc. The relevant n, P-values, and statistical analysis methods are indicated in each figure legend.

### 2. Results

VC Rescued Loss of DA Neurogenic Potential During In Vitro Expansion of VM-NSCs

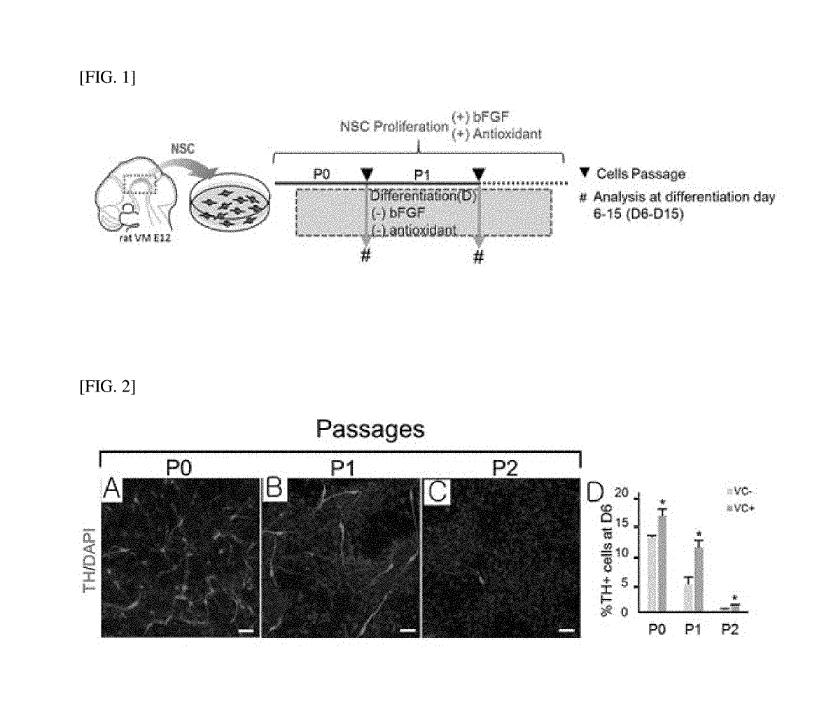

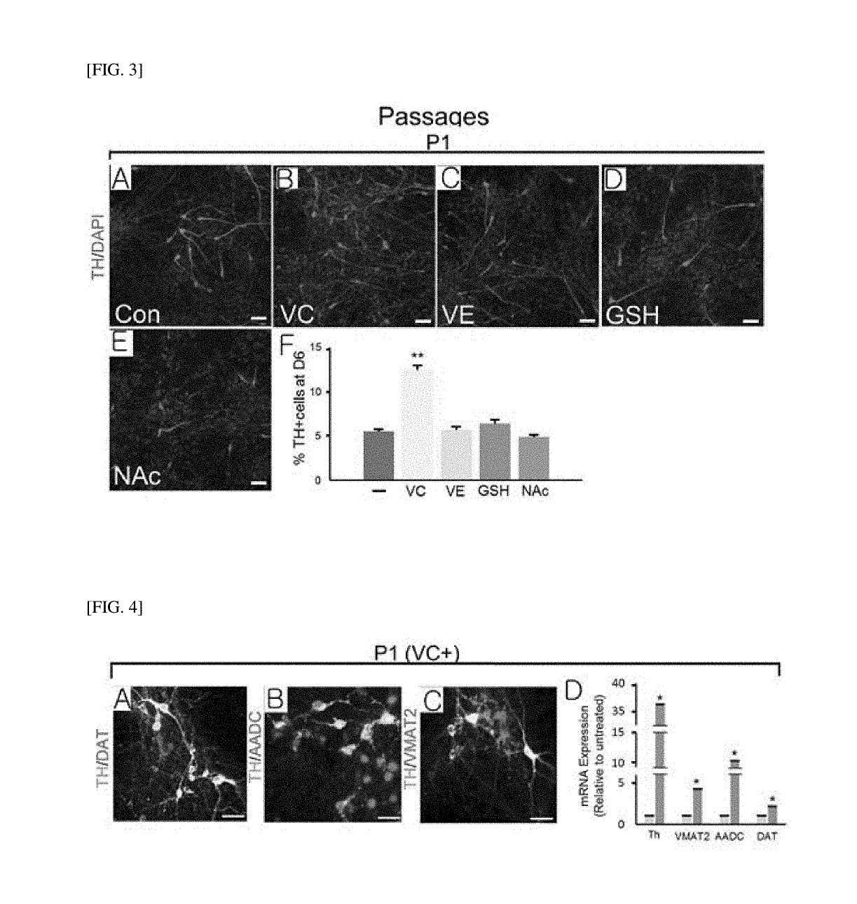

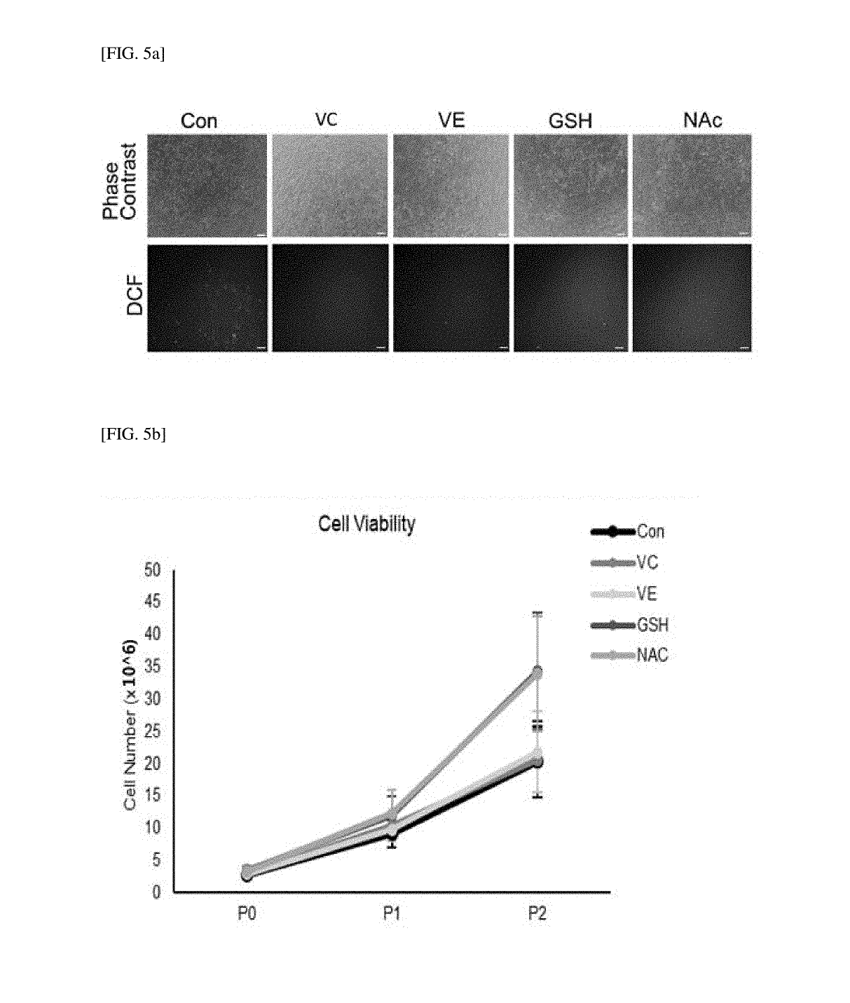

In stem cell-based treatment for PD, DA neurogenic potential is the most important. NSCs were isolated from naive rat embryo dopaminergic VMs at embryonic day (E12), and expanded in the presence of the mitogen basic fibroblast growth factor (bFGF) in vitro. Proliferating VM-NSCs were passaged every 4 days, and cells at each passage were induced to differentiate by withdrawing the mitogen (FIG. 1). The VM-NSCs expanded for a short period (4 days, unpassaged, P0) efficiently differentiated into DA neurons expressing tyrosine hydroxylase (TH), the key enzyme for DA biosynthesis; however, in order for VM-NSCs to become a systemic source of cells, more cells should be obtained, and when VM-NSC expansion is induced over cell passages for a longer period to this end, the differentiation potential from long-term expanded VM-NSCs into DA neurons was rapidly reduced (A-D of FIG. 2). Altered cellular characteristics, including decreased differentiation potential during in vitro expansion, are commonly observed in stem/precursor cell cultures derived from mammalian tissues, regardless of tissue origin. This may be regarded as a phenomenon associated with cellular aging, given that similar changes in stem cell properties are manifest during in vivo aging (based on the comparison of tissue-specific stem cells in young vs aged tissues). As reactive oxygen species (ROS) are a major cause of cellular aging and senescence, we tested whether elimination of ROS by anti-oxidant treatment in VM-NSC cultures could rescue the culture-dependent loss of DA neurogenic potential. To this end, the anti-oxidants vitamin C (VC), vitamin E (VE), reduced glutathione (GSH), or N-acetylcysteine (NAc) were used as supplements during in vitro VM-NSC expansion and subsequent culture differentiation was induced without the antioxidants (FIG. 1). ROS scavenging effects of all the anti-oxidants tested were confirmed by 2′,7′-dichlorofluorescin (DCF) staining (FIG. 5a). However, among the tested anti-oxidants, only treatment with VC resulted in the prevention of DA neuron loss in passaged cultures (A-F of FIG. 3), indicating that VC-specific actions, rather than general anti-oxidant effects, were responsible for the observed VC effect. VC treatment during NSC expansion was dramatic in cultures up to passage 1 (P1) which yielded P1 cultures, in which the TH+ cell yields (10.85±1.2% of total DAPI+ cells) were as great as those achieved in untreated cultures at passage 0 (P0) (12.5%±0.35%) (D of FIG. 2). Virtually all the TH+ cells differentiated from VC-treated VM-NSCs at P1 expressed the other DA phenotypes (VMAT2, AADC and DAT) (A-C of FIG. 4) along with increases of their gene expression (D of FIG. 4), suggesting that VC treatment not only induced TH expression but exerted an effect on authentic DA neuron differentiation.

The dramatic effect of VC treatment was not sustained in cultures which had undergone one additional passage (at P2), in which TH+DA neuronal yields from VC-treated NSCs were also sharply reduced to 1.22±0.1%; this was however still significantly greater than the VC-untreated control (0.44±0.22%, p<0.05, n=3 independent cultures) (D of FIG. 2). However, regarding the TH+DA neuronal yields, it was determined that, in development of a stem cell therapeutic agent, the VC treatment effect is effective also for the P1 cultures, and thus the following experiments were carried out to observe a VC effect up to P1 culture.

Midbrain-Specific Marker Expression, Pre-Synaptic Function, and Toxic Resistance of mDA Neurons Differentiated from VC-Treated VM-NSCs

The effect of VC on DA neuron differentiation has been reported in previous studies [Bagga, V., Dunnett, S. B., and Fricker-Gates, R. A. (2008). Ascorbic acid increases the number of dopamine neurons in vitro and in transplants to the 6-OHDA-lesioned rat brain. Cell transplantation 17, 763-773; He, X. B., Kim, M., Kim, S. Y., Yi, S. H., Rhee, Y. H., Kim, T., Lee, E. H., Park, C. H., Dixit, S., Harrison, F. E., et al. (2015). Vitamin C facilitates dopamine neuron differentiation in fetal midbrain through TET1- and JMJD3-dependent epigenetic control manner. Stem Cells 33, 1320-1332; Lee, J. Y., Koh, H. C., Chang, M. Y., Park, C. H., Lee, Y. S., and Lee, S. H. (2003). Erythropoietin and bone morphogenetic protein 7 mediate ascorbate-induced dopaminergic differentiation from embryonic mesencephalic precursors. Neuroreport 14, 1401-1404; Lee, S. H., Lumelsky, N., Studer, L., Auerbach, J. M., and McKay, R. D. (2000). Efficient generation of midbrain and hindbrain neurons from mouse embryonic stem cells. Nat Biotechnol 18, 675-679; Yan, J., Studer, L., and McKay, R. D. (2001). Ascorbic acid increases the yield of dopaminergic neurons derived from basic fibroblast growth factor expanded mesencephalic precursors. Journal of neurochemistry 76, 307-311]. However, in addition to DA neuron yield, successful cell therapeutic outcome relies on the expression of midbrain phenotypes, neuronal maturation, presynaptic (neurotransmitter release) functions, and cell survival of the differentiated DA neurons. In order to investigate VC effects on those aspects, we applied VC during VM-NSC proliferation and followed by VC withdrawal during differentiation. The rationale for the VC withdrawal during differentiation is to closely mimic cell transplantation condition, where VC addition is only limited to donor cell preparation period (before cell transplantation), since proliferating NSC stage is regarded as the most appropriate stage for cell transplantation, given that extensive cell death is associated with transplanting differentiating/differentiated neurons.

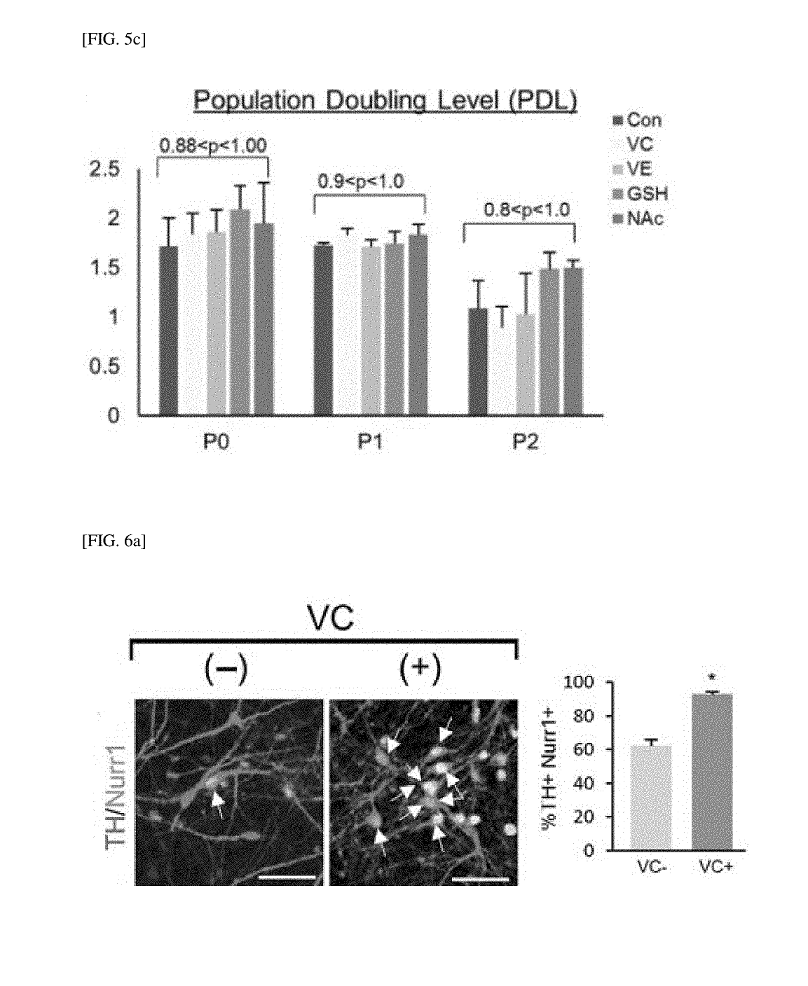

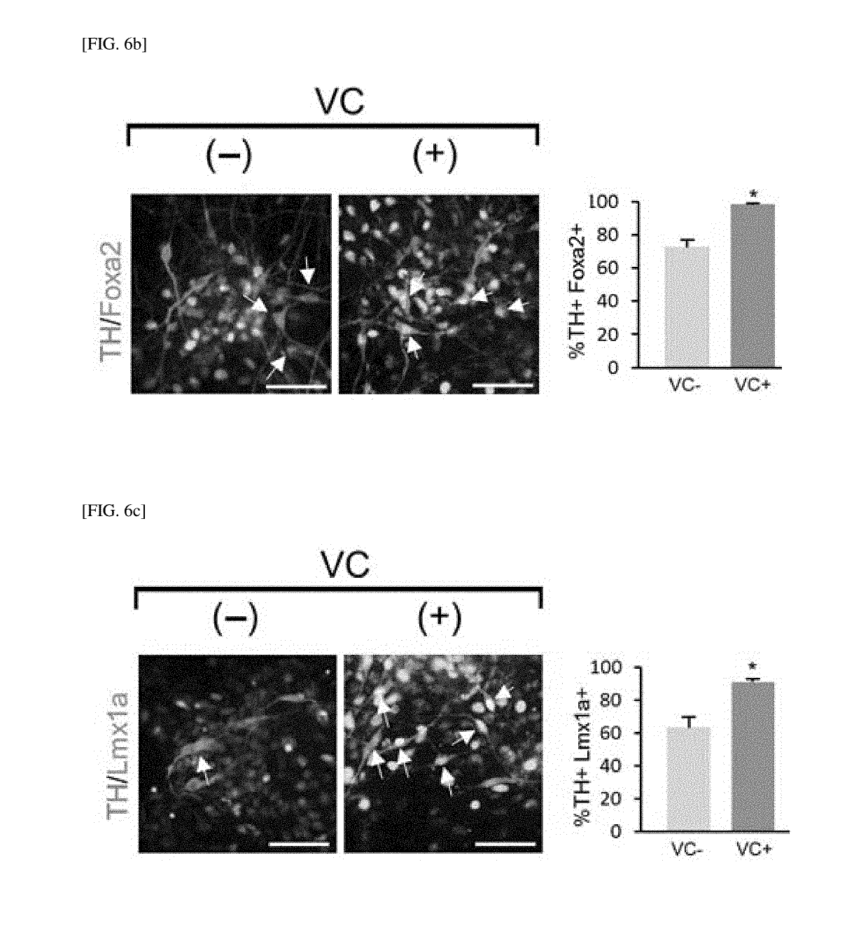

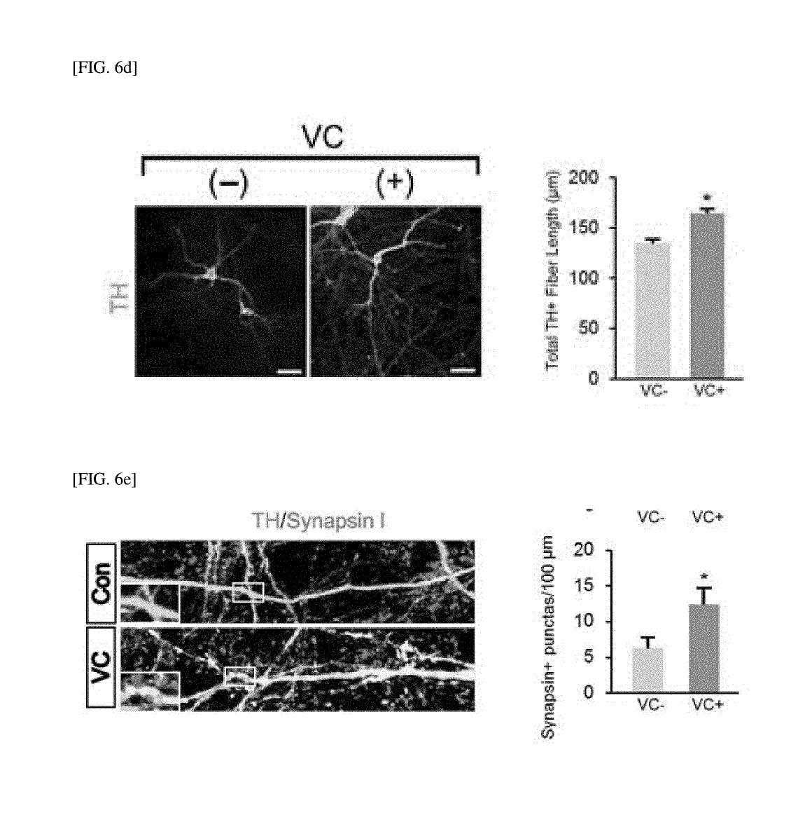

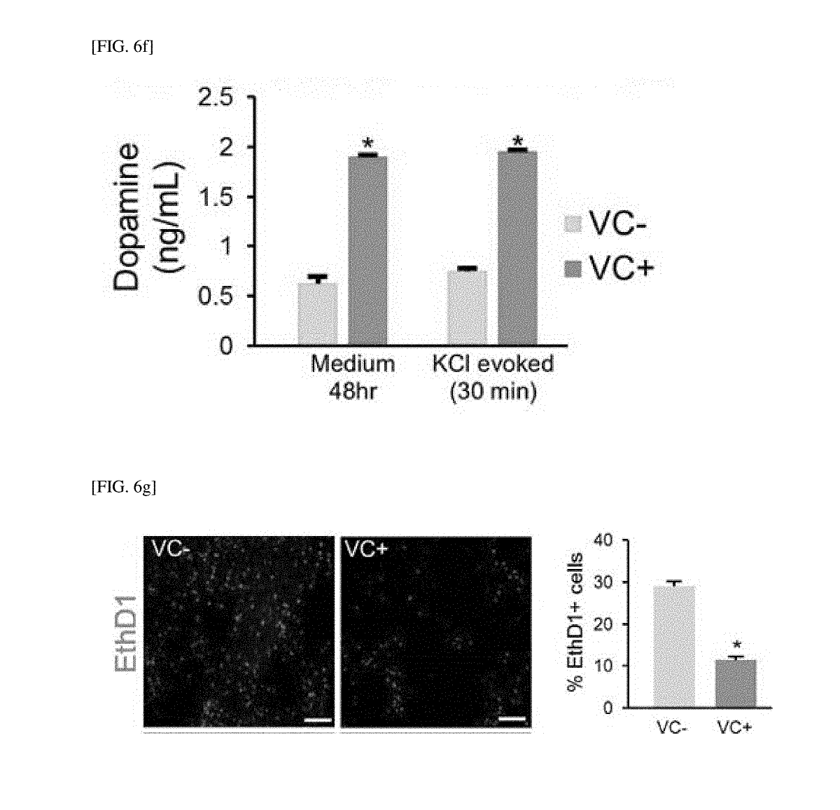

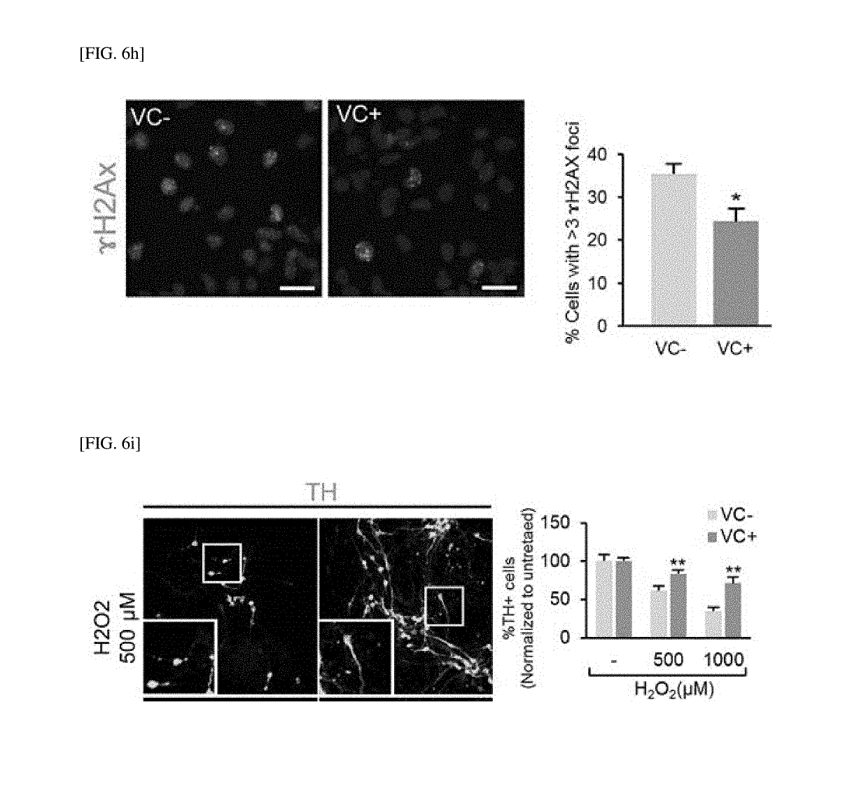

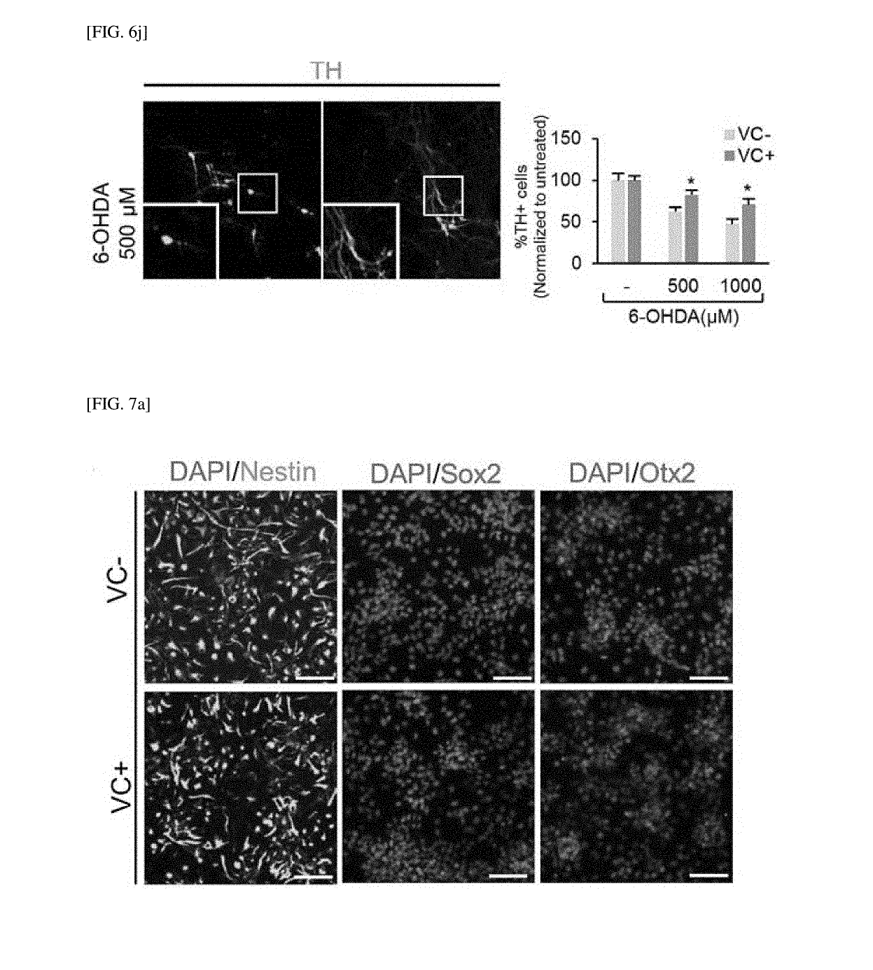

Midbrain-type DA (mDA) neurons are characterized by the continued expression of midbrain-specific developmental factors, such as Foxa2, Lmx1a, and Nurr1, after termination of development in the adult midbrain. Midbrain-specific factor expression in the mDA neurons is very critical for neuronal survival and function, and it has been known that the midbrain-specific factor easily disappears with aging and in unfavorable extracellular environments such as exposure to a toxin, and this is the reason why the PD easily occurs with aging and when exposed to a neurotoxin. Likewise, the loss of the midbrain-specific factor expression is shown in differentiated mDA neurons after long-term VM-NSC expansion and also in the mDA neurons survived in transplanted tissue, demonstrating that such loss is the important problem that should be solved to enable successful PD cell-based therapy. In a control in which differentiation was induced for 6 days without VC treatment performed during VM-NSC expansion to P1, only some of the differentiated TH+DA neurons showed midbrain-specific factor expression (midbrain-specific factors Nurr1 (62%), Foxa2 (73%) and Lmx1a (63%), FIGS. 6a to 6c). On the other hand, in P1 cultures treated with VC during expansion, DA neuronal yields were enhanced, and almost all TH+DA neurons expressed midbrain-specific factors (93% (Nurr1), 98% (Foxa2) and 91% (Lmx1a)).

In morphometric assessments, total fiber lengths per TH+DA neuron were significantly greater in cultures differentiated from VM-NSCs expanded with VC supplementation, compared to untreated control cultures (164 um vs 134 um, FIG. 6d). In images captured using confocal microscopy, the synaptic vesicle-specific markers synapsin+ puncta were more abundantly localized in TH+DA neuron neurites differentiated from VC-treated NSCs (FIG. 6e). VC treatment has also resulted in a significant increase in neuronal maturation in terms of synaptogenesis. We further observed that presynaptic DA neuronal function, as measured by DA release (FIG. 60, was greater in cultures differentiated from VC-treated NSCs. Taken together this indicates that maturity of DA neurons was morphologically, synaptically and functionally enhanced by VC treatment.

After long-term expansion and passaging of NSCs, cell death during or after differentiation increased, and the subsequent results show that cell death in the differentiated cells is considerably reduced by the VC treatment performed during the expansion and passaging. First, cell death was estimated by ethidium homodimer 1 (EthD-1)+ dead cell counting at day 8 (D8) of differentiation, and it was observed that cell death during or after differentiation was considerably reduced with the VC treatment performed during the NSC expansion and passaging (FIG. 6g). In addition, γH2AX foci, a DNA damage indicator observed during PD progression, were greatly reduced in VC treated differentiated cultures (% cells with >3 foci at D12: 35.5% vs 24.3%) (FIG. 6h). As expected, remarkably lower proportions of DA neurons died after exposure to toxic stimuli induced by H₂O₂ or 6-hydroxy DA (6-OHDA) in cultures differentiated from VC-treated NSCs (FIGS. 6i and 6j). In addition, TH+ cells that survived toxin treatment in VC-treated cultures displayed a healthy neuronal shape with extensive neurite outgrowth, while most of the surviving TH+ cells in the control cultures had blunted or fragmented neurites (insets of FIGS. 6i-6j), a neuronal aging and degenerative phenotype. We emphasize again that all the effects observed in the differentiated cultures were not directly mediated by VC, as the differentiated cells were cultured in the absence of VC.

VC-Mediated Epigenetic Control within a Range of mDA Neuron Developmental and Phenotype Genes

In view of VC's anti-oxidant action, mDA neuron differentiation enhanced by VC treatment could be attained by a selective mechanism, in which VC enhanced cell survival and proliferation selectively in DA neuronal lineage cells. However, NSC cultures derived from early rat embryonic brains are highly and sufficiently proliferative and viable in the presence of the mitogen bFGF. Thus, none of the anti-oxidant treatments tested, including VC, significantly altered VM-NSC survival and proliferation up to P1 (the stage during which all analyses in this study were done) (FIGS. 5b and 5c), ruling out the possibility of a selective mechanism in VC-mediated DA neuron differentiation.

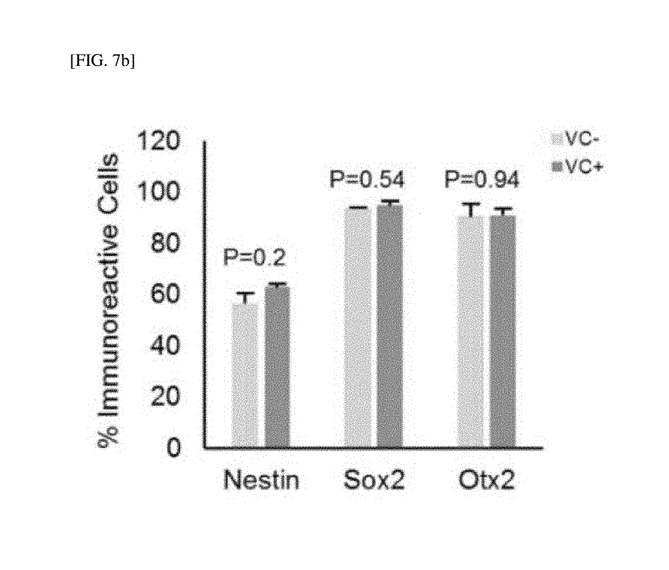

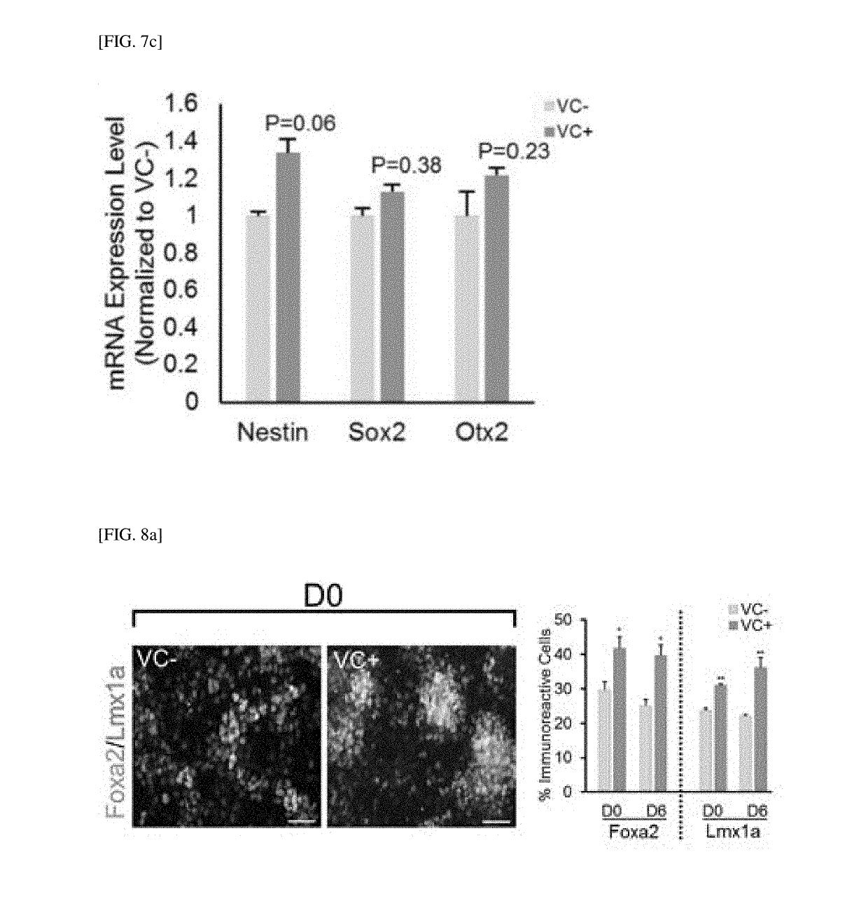

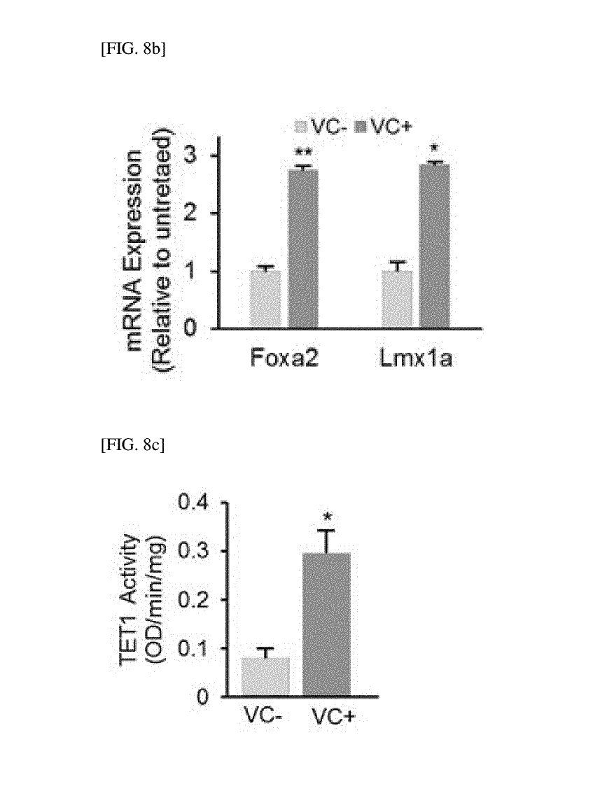

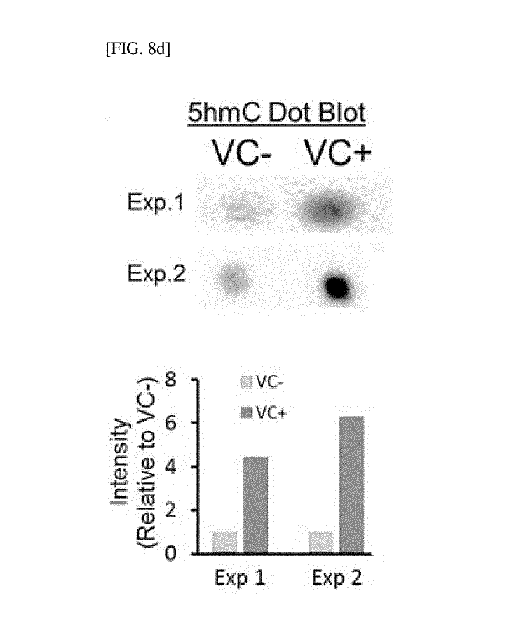

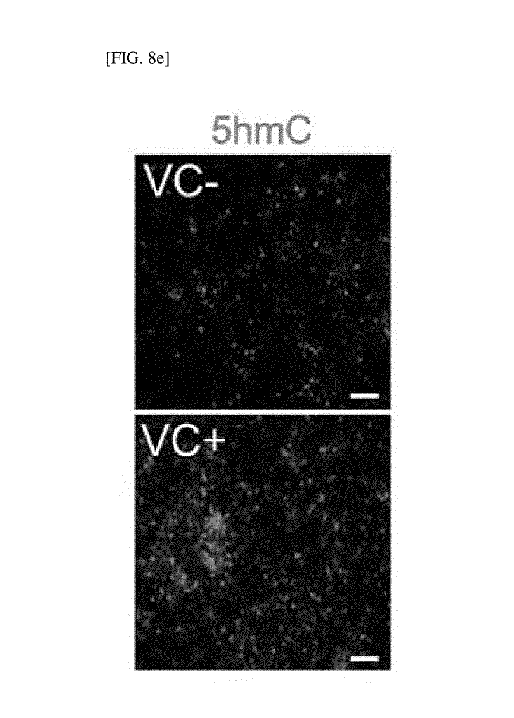

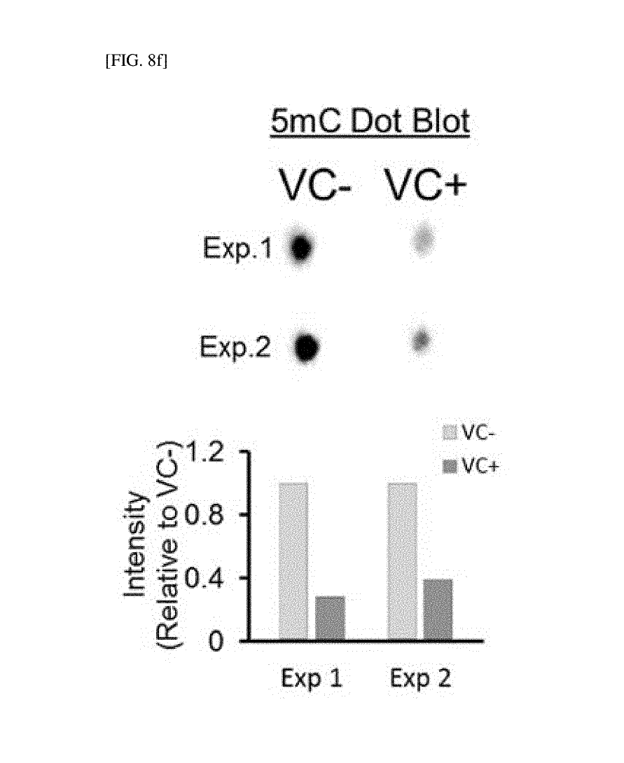

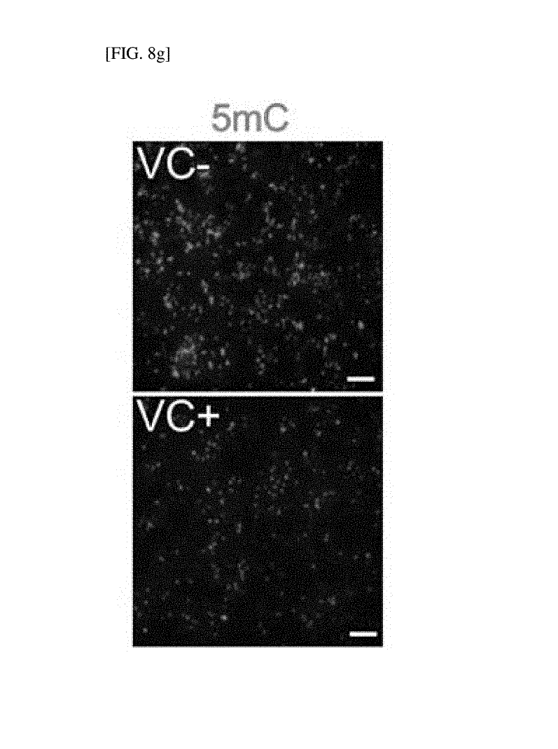

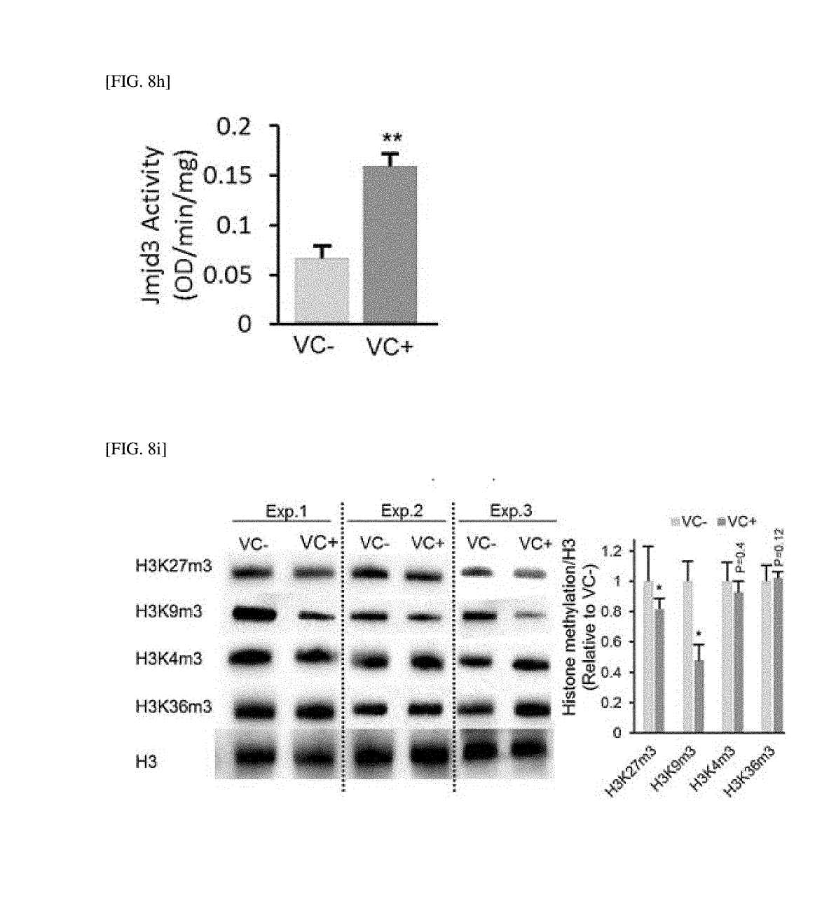

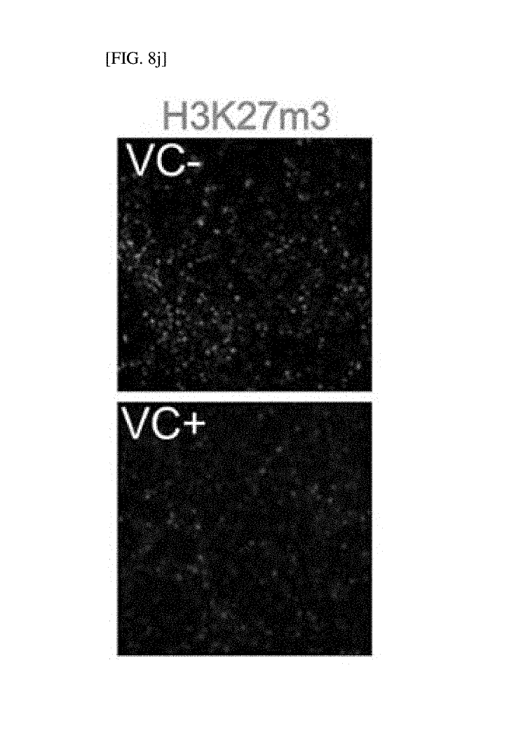

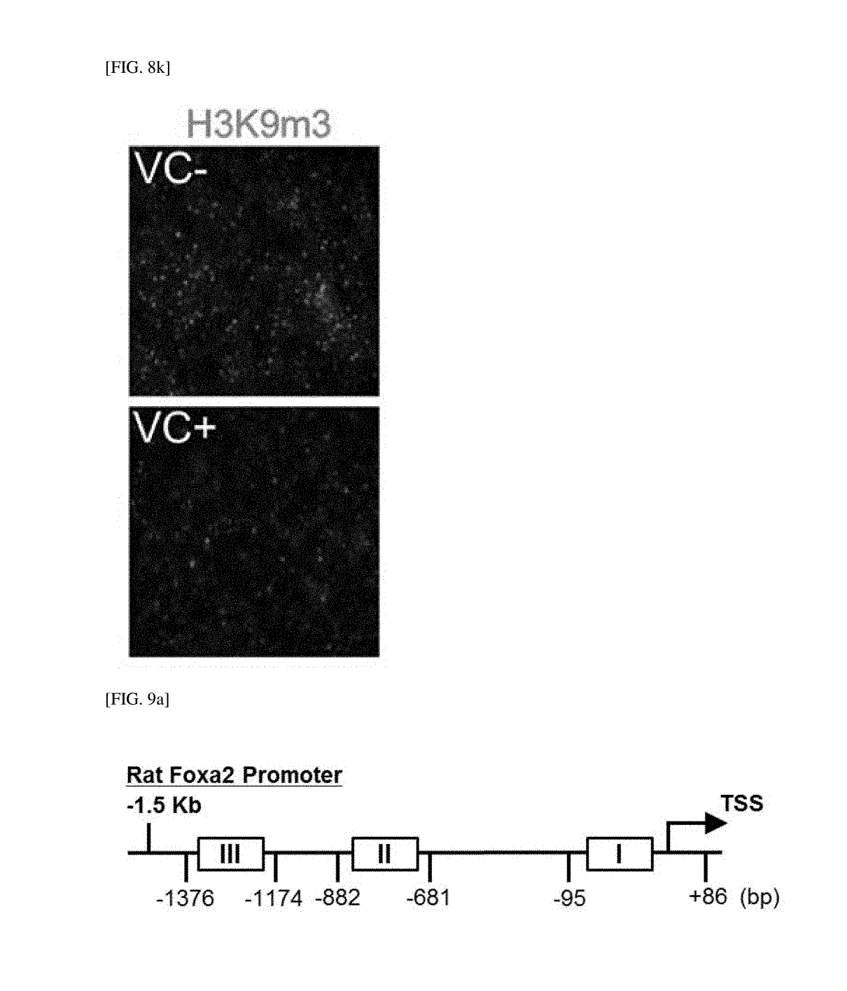

Expression of general NSC-specific (Nestin, Sox2) and anterior brain region-specific (Otx2) markers was not altered by VC treatment of proliferating VM-NSC cultures (FIGS. 7a-7c). By contrast, VC treatment during VM-NSC expansion enhanced the percentage of cells expressing the VM-specific NSC markers Foxa2 and Lmx1a at differentiation day 0 (D0, before differentiation induction) (FIG. 8a). Foxa2 and Lmx1a are expressed in dopaminergic NSCs from early developing VMs and act as the master regulators for mDA neuron development by inducing expression of a battery of later developmental genes, such as Nurr1, Pitx3, and neurogenin2 (Ngn2). In addition, as mentioned earlier, expression of Foxa2 and Lmx1a continued in adult mDA neurons was critical for survival, phenotype maintenance, and functions of this neuronal type. Of note, the percentage of Foxa2+ and Lmx1a+ cells increased by VC treatment during the NSC stage was sustained after differentiation (after VC withdrawal) (FIG. 8a graph); this was directly associated with increased Foxa2/Lmx1a expression in differentiated DA neurons (FIGS. 6b-6c) and, thus, enhanced DA neuron survival and presynaptic function (FIGS. 6f-6j).

Next, we sought to determine how VC promoted Foxa2 and Lmxa1 expression in proliferating VM-NSCs. Quantitative real-time PCR analyses showed VC treatment enhanced the mRNA levels of Foxa2 and Lmx1a (FIG. 8b), indicating that VC acted at the gene transcription level. In addition to its anti-oxidant role, VC acts as a cofactor for the family of Fe(II)-2-oxoglutarate-dependent dioxygenases, including epigenetic control enzymes such as ten-eleven-translocation 1-3 (Tet1-3) and Jumonji C (JmjC)-domain-containing histone demethylases (Jmjds). VC treatment of proliferating VM-NSCs greatly enhanced Tet enzyme activity in nuclear fractions (FIG. 8c), with which methylated cytosine on CpG sites (5-methylcytosine; 5mC) of DNA is hydroxylated into 5-hydoxymethylcytosine (5hmC). Consequently, global 5hmC levels estimated by immunoblotting (FIG. 8d) and immunocytochemical (FIG. 8e) analyses were greatly increased in VC-treated VM-NSCs; this was accompanied by a significant decrease in global 5mC levels (FIGS. 8f and 8g). The increase in Jmjd enzyme activity was observed in the nuclear fractions of VC-treated VM-NSCs (FIG. 8h). Among the histone methylations tested, global levels of H3K27m3 and H3K9m3 were reduced by VC treatment (FIGS. 8i-8k).

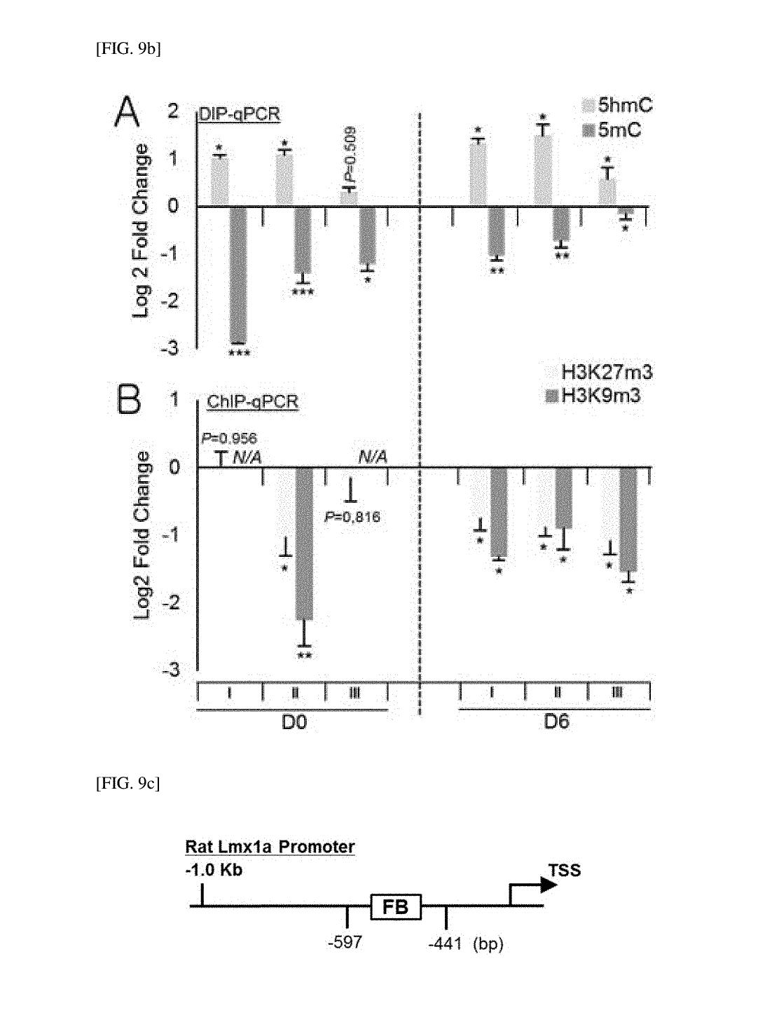

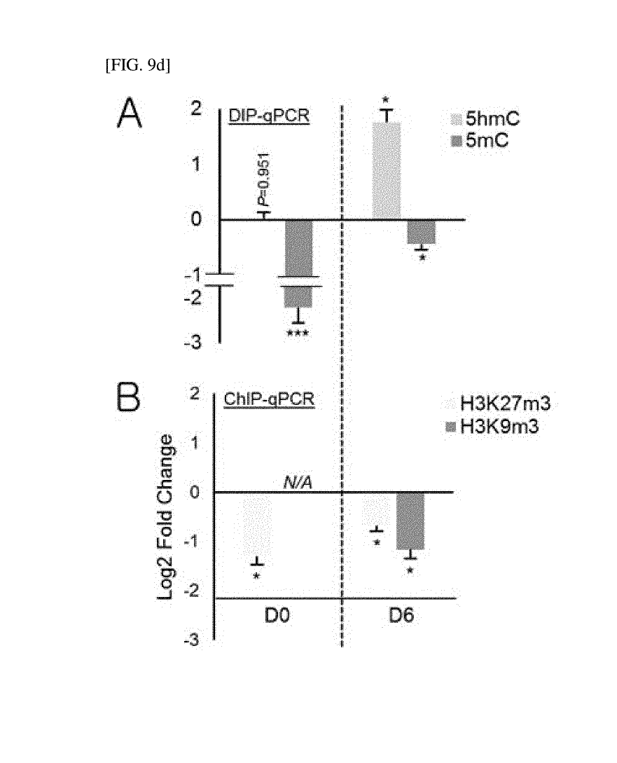

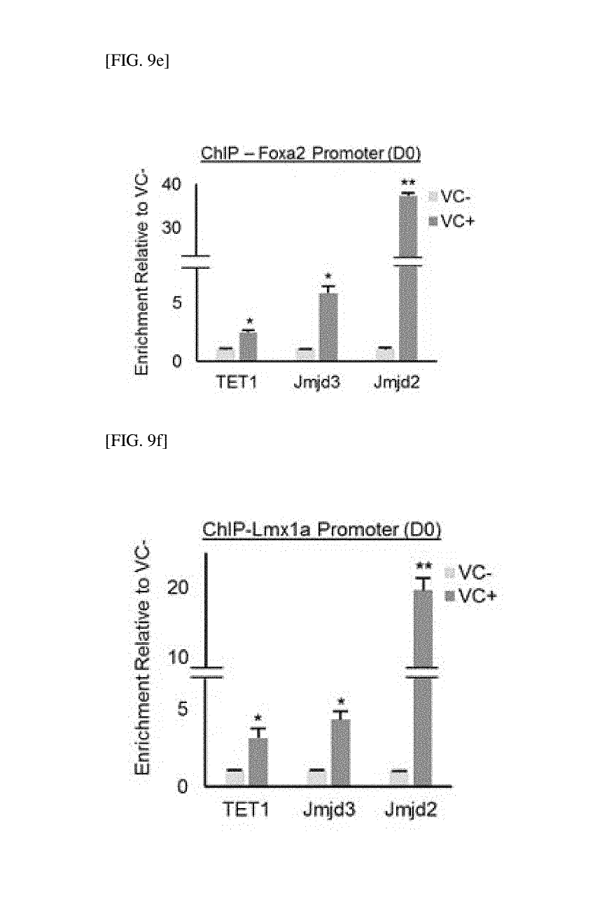

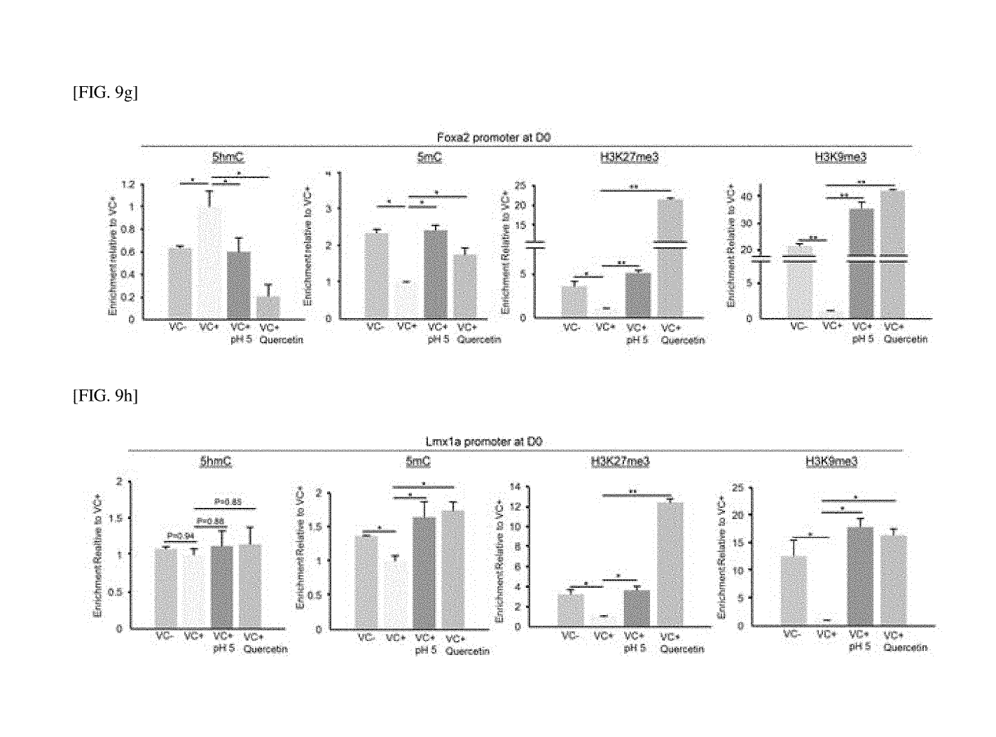

Based on these findings, we assessed 5hmC/5mC and H3K27m3/H3K9m3 levels in the promoter regions of Foxa2 and Lmx1a (FIGS. 9a and 9c). hMeDIP-qPCR and MeDIP-qPCR analyses revealed that in the Foxa2 promoter regions 5hmC was more abundant in VC-treated NSCs, while 5mC levels were reduced (FIGS. 9b and 9d). In addition, increased binding of Tet1, Jmjd3 and Jmjd2 proteins to Foxa2 and Lmx1a promoter regions in VC-treated VM-NSCs was confirmed by ChIP-qPCR analysis (FIGS. 9e and 9f), indicating that epigenetic changes observed by epigenetic enzymes occur. In the developing brain, intracellular VC contents are much higher than in extracellular fluids due to the action of sodium-dependent VC transporter 2 (SVCT2) transporting VC into the intracellular space against its gradient in a sodium- and pH-dependent manner. Blocking SVCT2 action by lowering pH or by treatment with quercetin, a specific SVCT2 inhibitor, abolished the VC-mediated changes to 5mC/5hmC and H3K27m3/H3K9m3 on the Foxa2 and Lmx1a genes (FIGS. 9g and 9h). These findings collectively demonstrated the direct action of VC on observed epigenetic changes. Collectively, with VC treatment, VC is transported into VM-NSC cells and promotes TET and Jmjd2/3 activities in the nucleus to induce the observed changes to 5mC/5hmC and H3K27m3/H3K9m3 on Foxa2 and Lmx1a promoters, indicating that these genes were epigenetically activated and their gene expression was increased. In addition, considering the actions of Foxa2 and Lmx1a as the master regulators of mDA neuron development, the VC-mediated epigenetic controls in those master genes are proposed to be the central mechanism for the effects of VC on enhanced mDA neuron differentiation. Interestingly, the VC-mediated changes in 5mC/5hmC and H3K27m3/H3K9m3 in Foxa2 and Lmx1a were maintained in the differentiated cells long after VC withdrawal (A,B of FIG. 9b and A,B of FIG. 9d). The sustained open DNA/chromatin structures of these genes likely contributed to sustained Foxa2 and Lmx1a expression in DA neurons in differentiated cultures, as shown in FIGS. 6b and 6c.

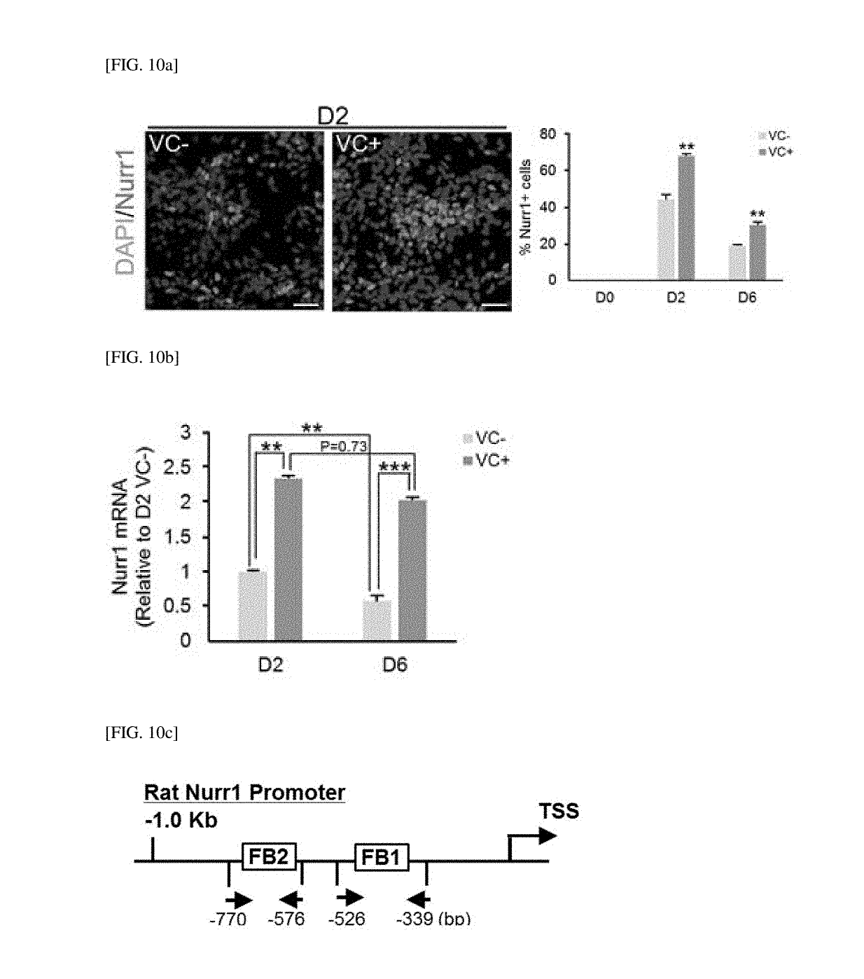

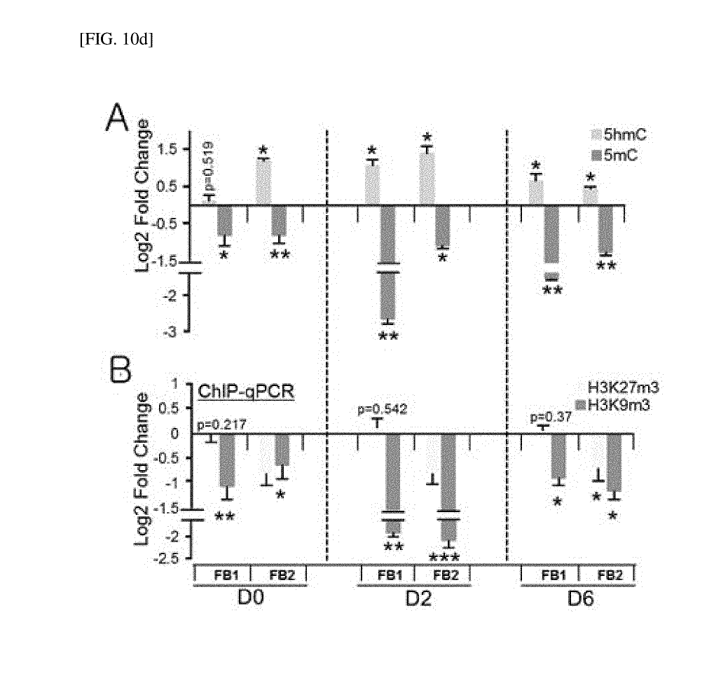

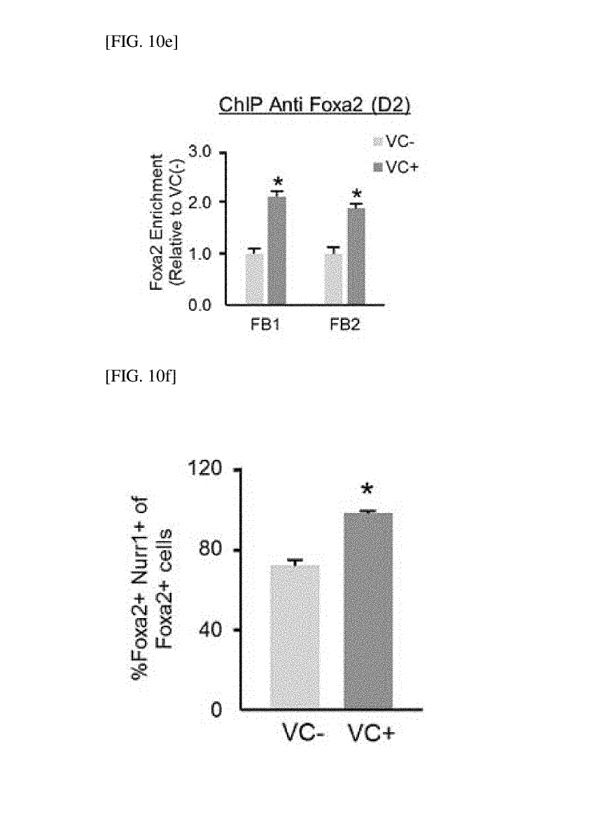

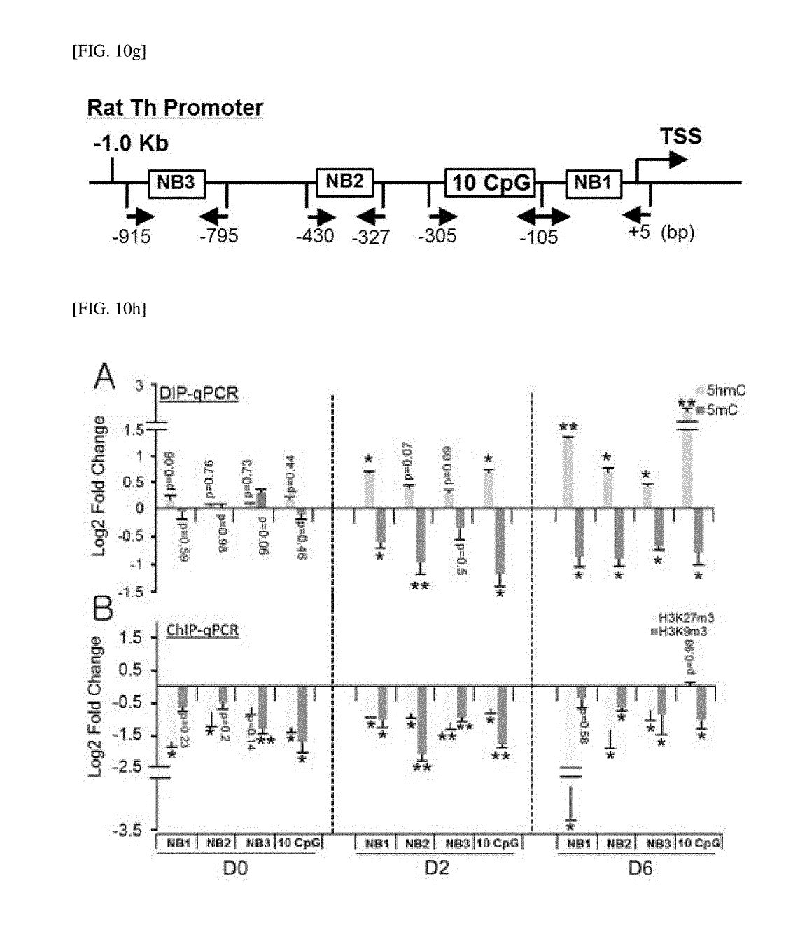

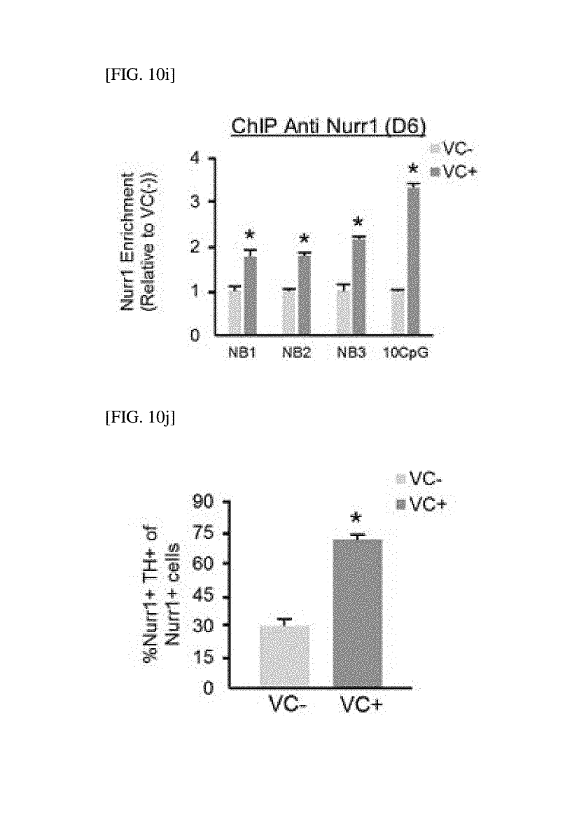

It has been reported that open epigenetic signatures on late developmental/differentiated phenotype genes are frequently established during an early stage of stem cell differentiation, without their actual expression. (Mikkelsen, T. S., Ku, M., Jaffe, D. B., Issac, B., Lieberman, E., Giannoukos, G., Alvarez, P., Brockman, W., Kim, T. K., Koche, R. P., et al. (2007). Genome-wide maps of chromatin state in pluripotent and lineage-committed cells. Nature 448, 553-560). Thus, we further examined the effect of VC on epigenetic changes in later mDA developmental and differentiated genes. Nurr1, the transcription factor critical for DA phenotype gene expression, begins to be expressed from late mDA neuron progenitor cells. Consistent with this, Nurr1-expressing cells were not detected in proliferating VM-NSC stages (D0), but began to be detected from differentiation day 2 (D2) (FIGS. 10a, 10b). The percentage of Nurr1+ cells at D2 was greater in cultures treated with VC during the proliferating NSC stage, and this increase in Nurr1 expression was sustained during the later differentiation period (FIGS. 10a, 10b). Interestingly, compared to untreated controls, increased 5hmC levels with 5mC decreases and the decrease of H3K27m3/H3K9m3 at Nurr1 promoter regions (consensus Foxa2 binding sites, FIG. 10c) were observed in the VC-treated cultures from the undifferentiated stage (at D0) (A, B of FIG. 10d) when no Nurr1 expression was detected (FIG. 10a, graph). These epigenetic code changes became greater at D2 and were maintained at least up to D6. Along with the activated epigenetic patterns associated with open DNA/chromatin structures, Foxa2, a transcription factor known to directly induce Nurr1 transcription, was more abundantly recruited to the Nurr1 promoter regions at D2 in the culture pre-treated with VC (FIG. 10e). Consequently, the percentage of Foxa2+ cells co-expressing Nurr1 at D2 was significantly greater in VC-treated cultures (FIG. 10f).

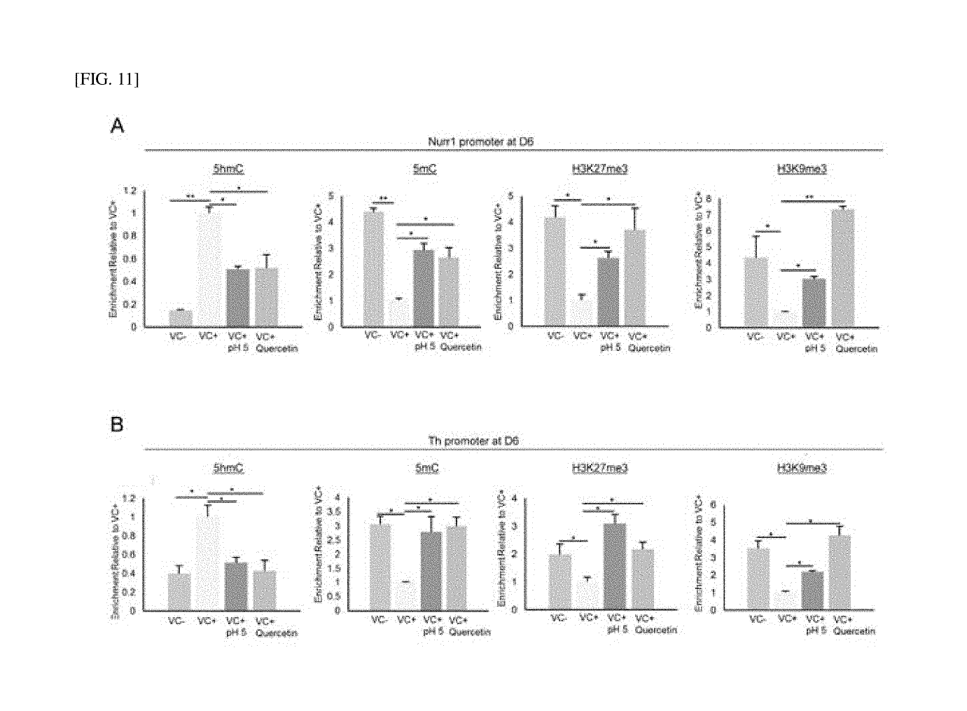

VC treatment during the proliferation period also induced similar epigenetic changes (except 5hmC/5mC at D0) on the TH promoter (FIG. 10g), a DA phenotype gene expressed in terminally differentiated DA neurons, from D0 (A, B of FIG. 10h). There was an increasing trend in the activated epigenetic code levels, especially in 5hmC at TH promoters, during the 6 days of differentiation. Consistent with this, ChIP-qPCR analyses showed significantly greater Nurr1 protein abundance at the consensus Nurr1 binding sites and a CpG-enriched site (close to the TSS) of the TH promoter at D6 in VC-treated cultures (FIG. 10i), resulting in an enhanced ability of Nurr1 to induce TH expression, estimated by the percentage of Nurr1+, TH+ cells among total Nurr1+ cells upon VC treatment (FIG. 10j). Again, the VC-induced epigenetic changes to the Nurr1 and TH gene promoters were abolished by blocking SVCT2 activity (FIG. 11).

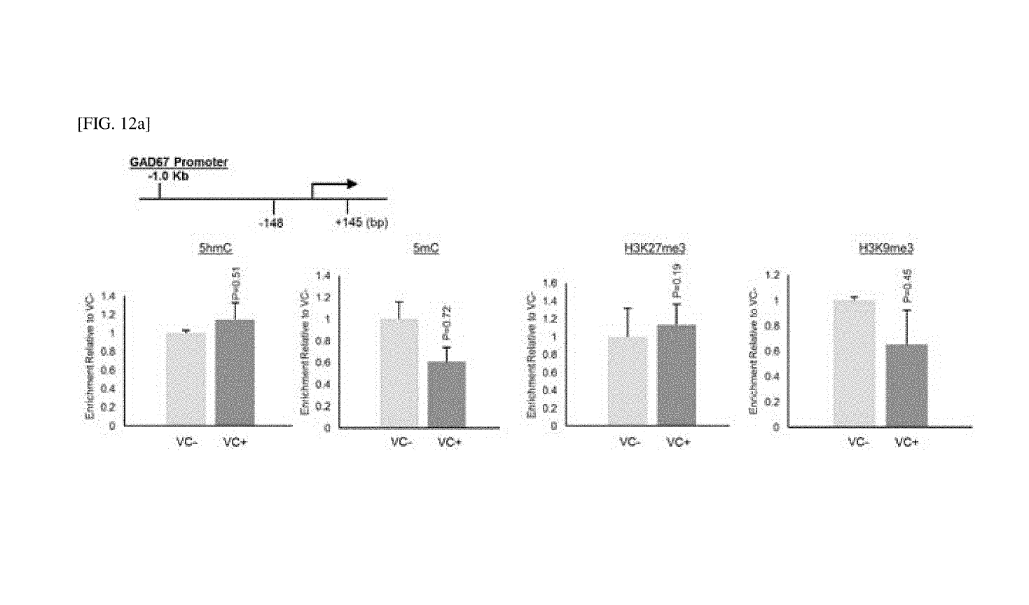

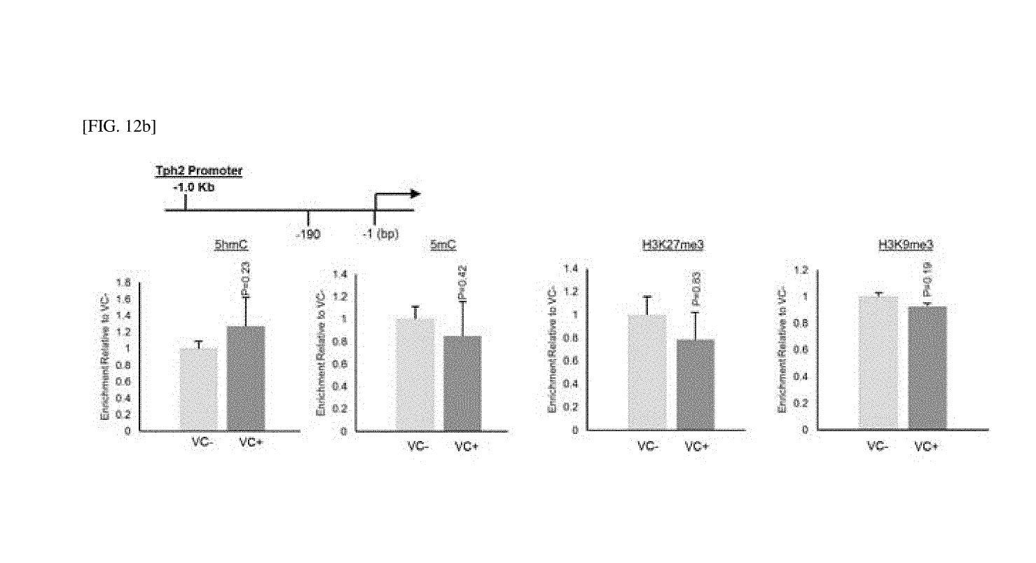

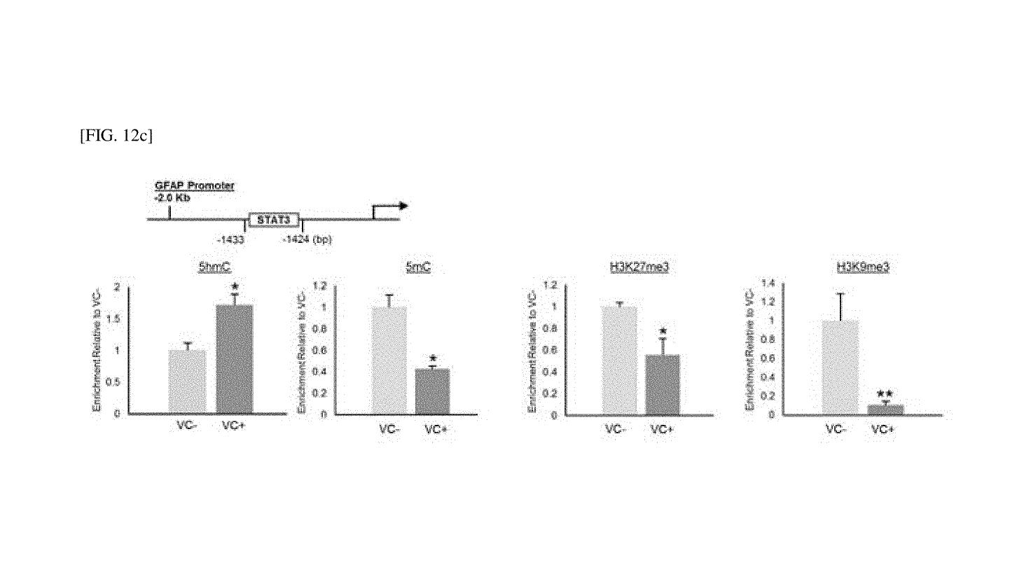

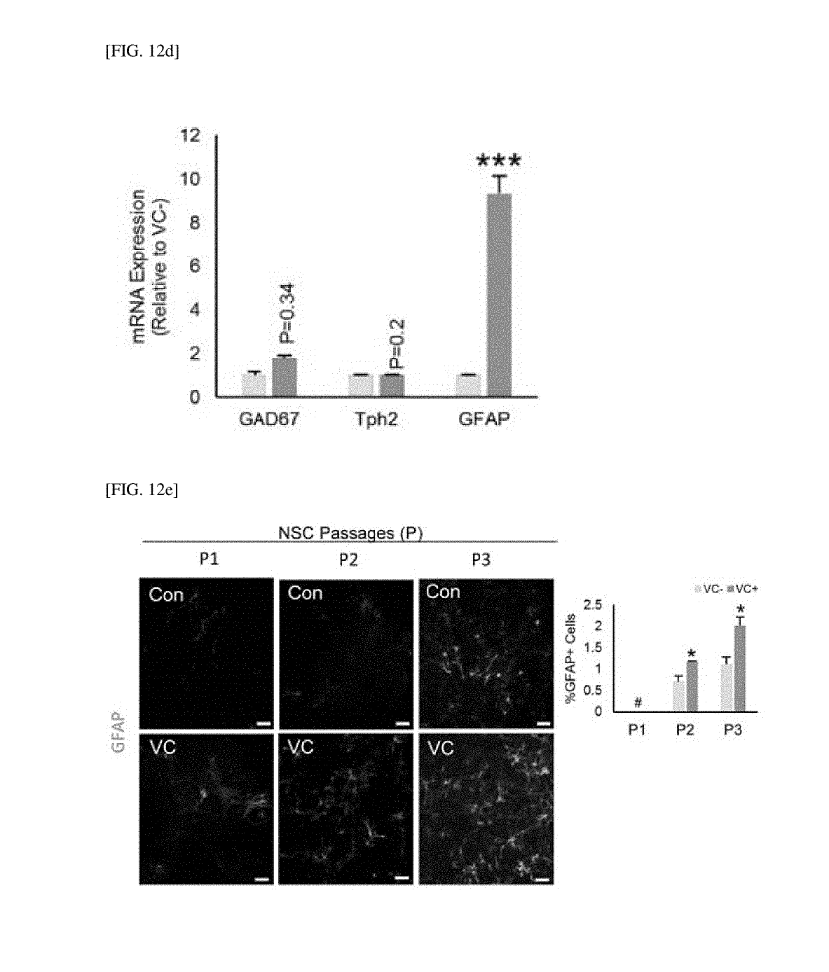

VC administration to proliferating VM-NSCs did not affect the epigenetic codes on the other neuronal subtype genes such as tryptophan hydroxylase 2 (TPH2, serotonergic neurons) and glutamic acid decarboxylase 67 (GAD67, GABAergic)(FIGS. 12a, 12b) along with no significant alteration in their mRNA expression levels (FIG. 12d). It means that the VC-mediated epigenetic gene expression is specifically observed only in mDA neurons. By contrast, VC-mediated changes to 5hmC/5mC and H3K27m3/H3K9m3 levels observed in the TH promoter were similarly detected in the promoter of glial fibrillary acidic protein (GFAP), specific for astrocytes (consensus STAT-binding region)(FIG. 12c). Consistently, mRNA expression of GFAP as well as GFAP+ astrocyte yields were significantly enhanced by treatment with VC during the proliferating VM-NSC stage (FIGS. 12d, 12e). VC effects on astrocyte differentiation are further addressed in the Discussion section. In summary, we showed that VC treatment of proliferating VM-NSCs induced epigenetic changes into open DNA/chromatin structures in a range of DA neuron developmental genes specific to early (Foxa2, Lmx1a), intermediate (Nurr1) mDA neuron progenitors and terminally differentiated DA neurons (TH). The VC-mediated epigenetic changes in these genes were sustained long after VC withdrawal, including in terminally differentiated mDA neurons. Thus, sustained epigenetic changes resulting from transient VC treatment of proliferating VM-NSCs were responsible for enhanced DA neurons differentiation and sustained midbrain-specific factor expression in differentiated mDA neuronal cells.

Cell Transplantation in PD Rats

Based on the in vitro findings, we ultimately assessed the therapeutic functions of VM-NSCs expanded with VC supplementation in a PD animal model.

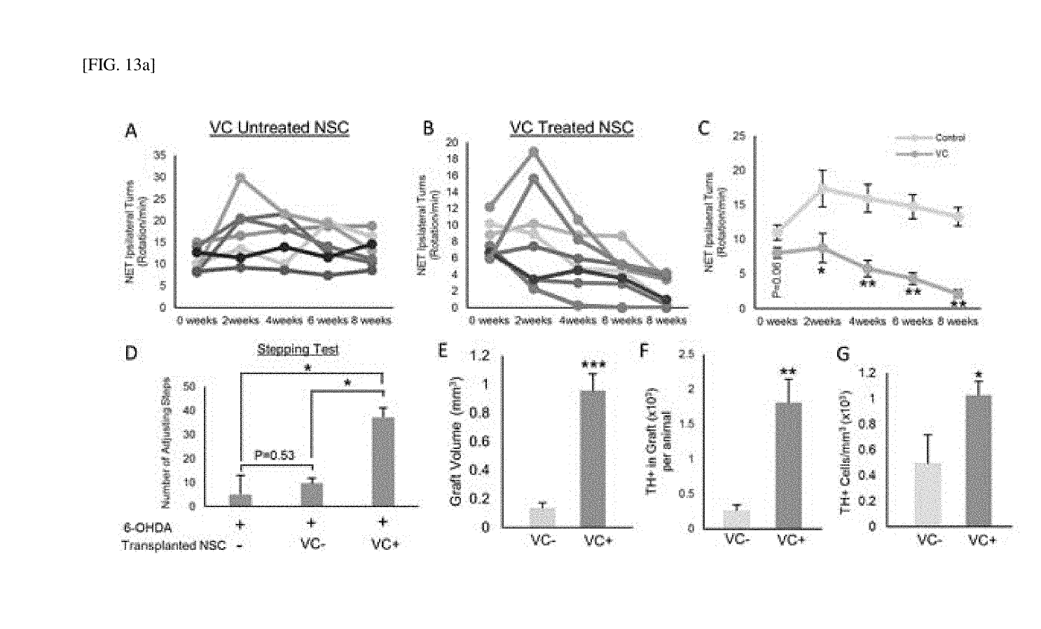

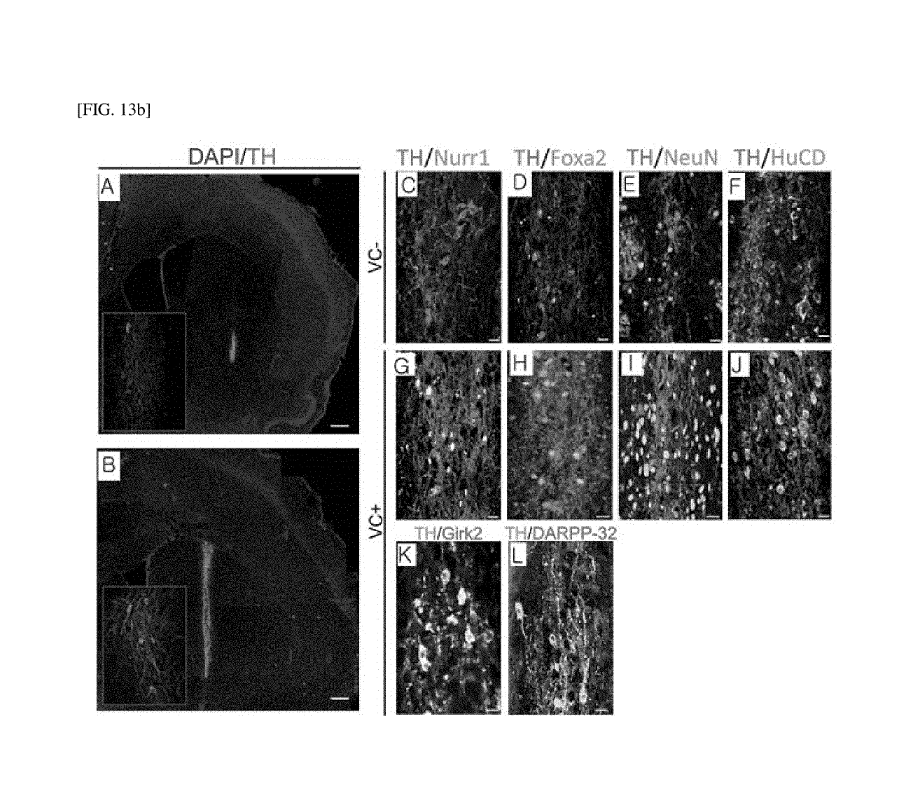

To do this, rat E12 VM-NSCs were expanded in vitro for 8 days (passaged at day 4 of proliferation) in the presence or absence of VC (P1D0), harvested and intrastriatally transplanted into a hemi-parkinsonian rat model. While functional recovery of PD rats has been demonstrated using transplantation of short-term expanded NSCs derived from early embryonic VM tissues. [Jensen, P., Pedersen, E. G., Zimmer, J., Widmer, H. R., and Meyer, M. (2008). Functional effect of FGF2- and FGF8-expanded ventral mesencephalic precursor cells in a rat model of Parkinson's disease. Brain research 1218, 13-20; Kim, J. Y., Koh, H. C., Lee, J. Y., Chang, M. Y., Kim, Y. C., Chung, H. Y., Son, H., Lee, Y. S., Studer, L., McKay, R., et al. (2003). Dopaminergic neuronal differentiation from rat embryonic neural precursors by Nurr1 overexpression. Journal of neurochemistry 85, 1443-1454.; Studer, L., Tabar, V., and McKay, R. D. (1998). Transplantation of expanded mesencephalic precursors leads to recovery in parkinsonian rats. Nat Neurosci 1, 290-295; Timmer, M., Grosskreutz, J., Schlesinger, F., Krampfl, K., Wesemann, M., Just, L., Bufler, J., and Grothe, C. (2006). Dopaminergic properties and function after grafting of attached neural precursor cultures. Neurobiol Dis 21, 587-606], this would not be expected to be achieved by transplanting VM-NSCs expanded and passaged for a longer period without VC treatment due to the loss of DA neurogenic potentials, loss of midbrain-specific factor expression and poor cell survival during and after differentiation. As expected, an amphetamine-induced rotation test revealed no significant reduction in rotation scores compared to pre-transplantation values in PD rats grafted with control NSCs (n=7). In contrast, dramatic behavioral recoveries were achieved in animals grafted with VM-NSCs expanded in the presence of VC. In all 8 rats grafted with VC-treated NSCs, without exception, rotations were reduced at 8-weeks post-transplantation (NET rotations on average; 2.1±0.6 (VC) vs 13.3±1.3 (control) rotation per minutes; A-C of FIG. 13a). We also assessed PD behaviors using non-pharmacological assays at 8-weeks post-transplantation. In the stepping test (D of FIG. 13a), rats grafted with VC-treated NSCs used their lesioned (left) forelimbs more often than did rats grafted with control-NSCs. The percentage of adjusting steps of the lesion containing paw was 37.3±3.9 (VC) % vs 9.7±2.2% (Control) of the right unlesioned paw, n=7 (Control) and n=8 (VC), p=7.3e-8, ANOVA followed by Tukey's post hoc analysis, (D of FIG. 13a).

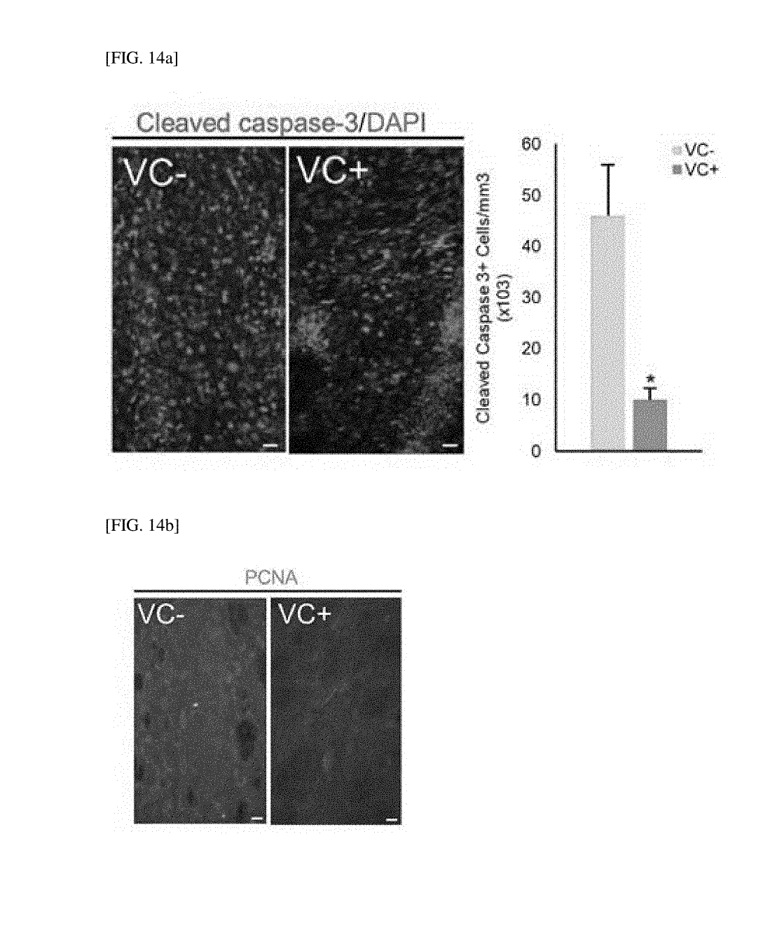

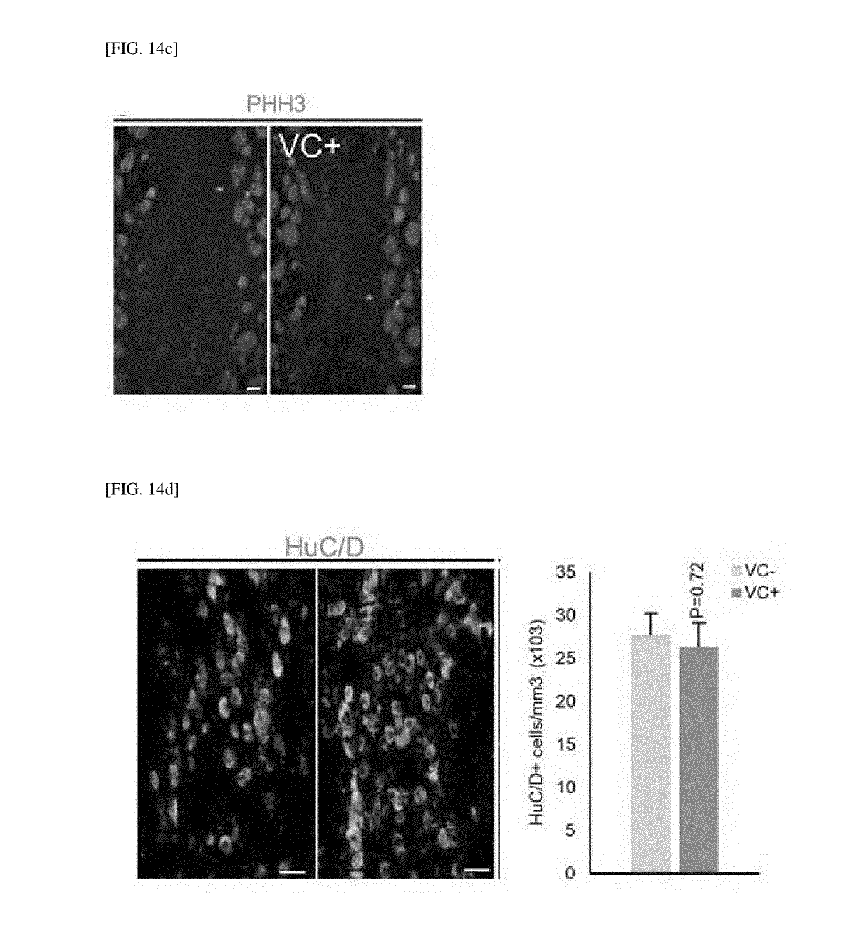

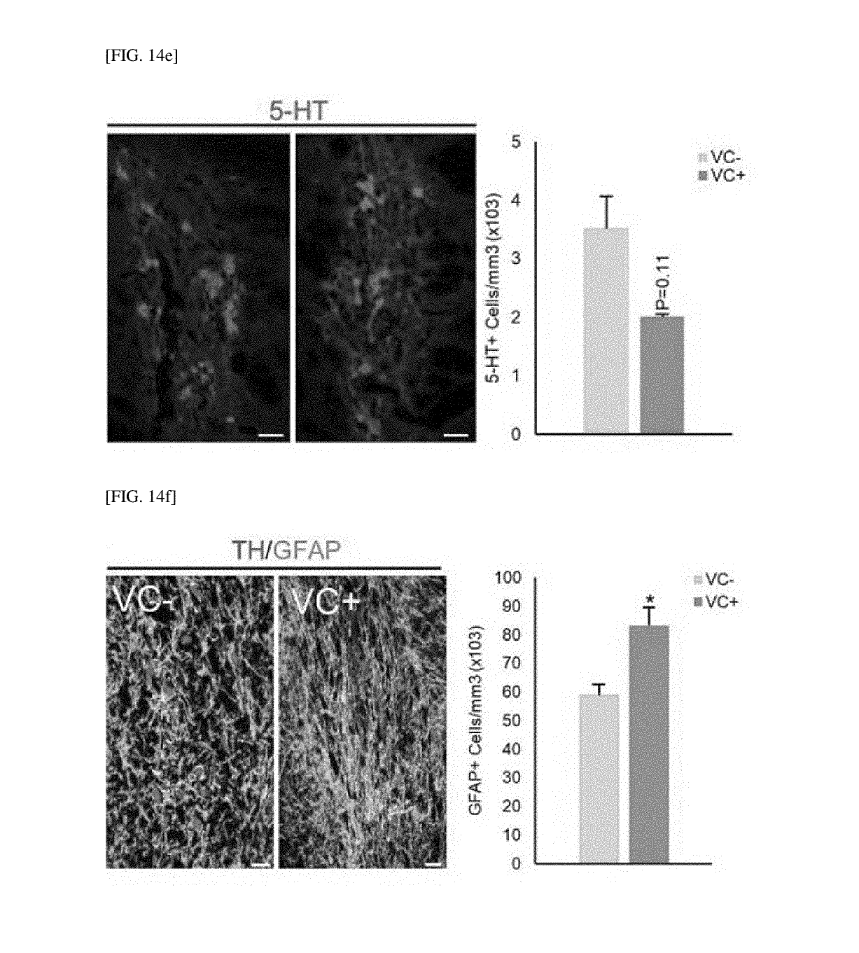

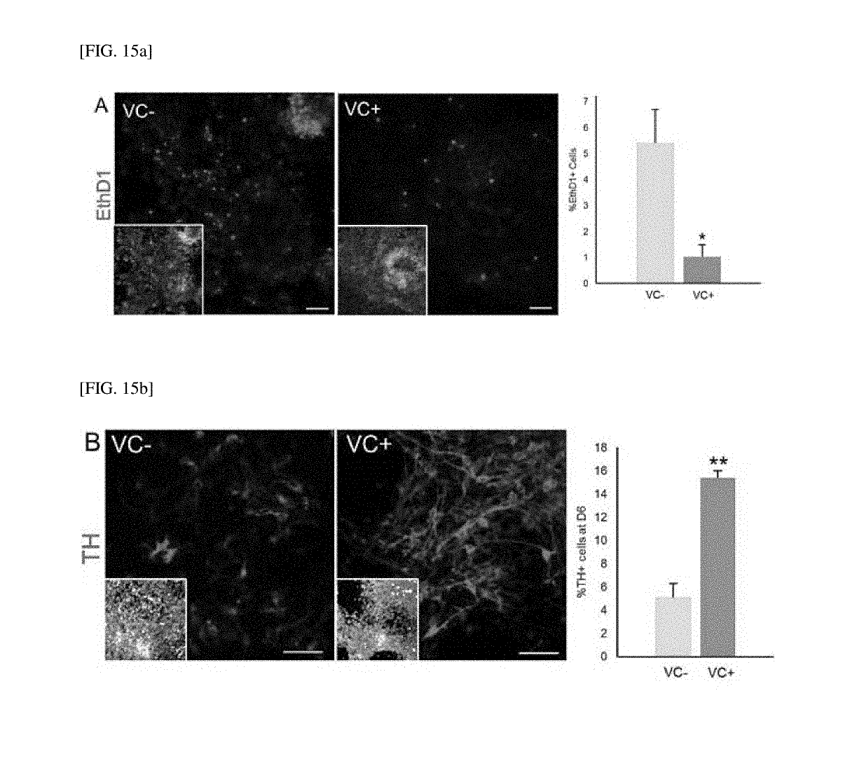

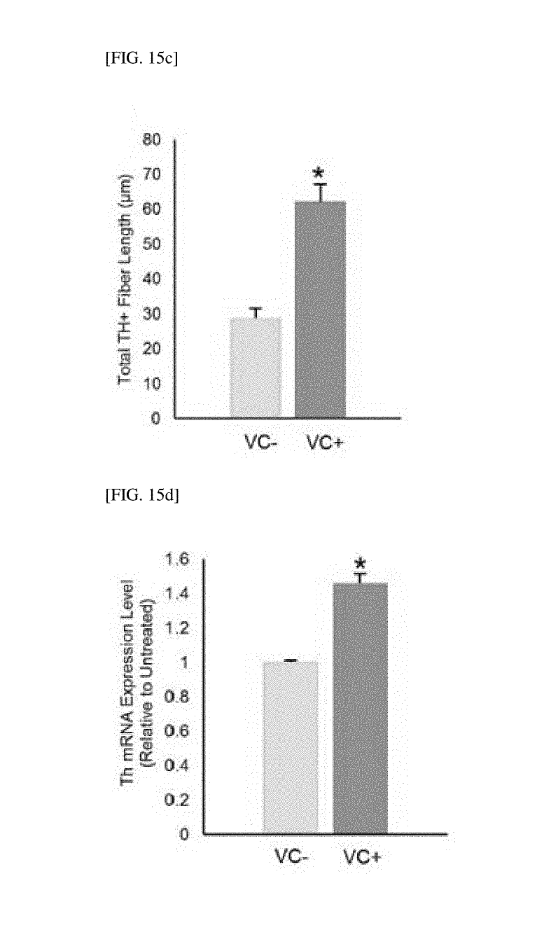

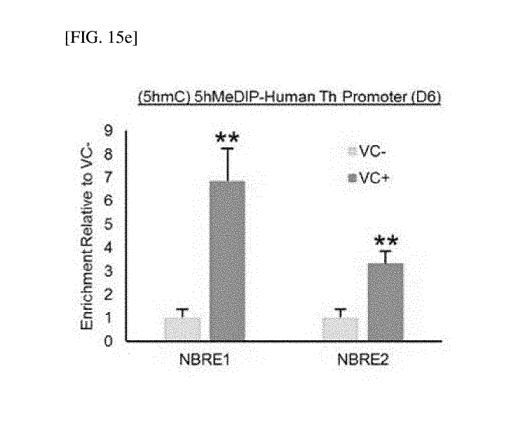

Consistent with the observed increased survival of differentiating/differentiated cells from VC-treated NSCs in vitro, histological analyses performed 8 weeks post-transplantation exhibited much larger graft formation in rats transplanted with VC-treated NSCs than control NSCs. (0.95±0.11 mm³ in VC group, n=8 vs 0.13±0.04 in the control group, n=7, p=1.4 e-4, Student's t-test, E, G of FIG. 13a, A, B of FIG. 13b). Consistently, more abundant cleaved caspase 3+ apoptotic cells in grafts were detected in the control group compared to the VC group (FIG. 14a). None in the control and VC-treated NSC grafts were positive for the proliferating cell markers of proliferating cell nuclear antigen (PCNA) and phospho-histone H3 (pHH3) (FIGS. 14b, 14c). There were 1802±338 TH+ cells (VC) vs 260±78 TH+ cells (control) in the graft per animal (F of FIG. 13a). However, the cell densities of general neuronal (HuC/D+, FIG. 14d) and serotonergic neuronal cells (5-hydroxy-tryptamine, 5-HT+, FIG. 14e) were not significantly different between the control and VC grafts. Consistent with the observed in vitro VC effects on astrocytic differentiation (FIGS. 12d, 12e), GFAP+ astrocytes were greater in the grafts generated by VC-treated NSCs (FIG. 140. TH+ cells in the VC-treated NSC grafts exhibited more mature neuronal shapes with multiple and extensive neurite outgrowths than those in the control grafts (A, B of FIG. 15b, insets). In addition, there was a very clear difference in the co-expression of markers specific for mature neurons and mDA neurons in TH+ cells, where in the control grafts virtually none or a few of the TH+ cells were positive for mature neuronal and mDA neuronal markers (C-F of FIG. 13b). In clear contrast, expression of mature neuronal (NeuN and HuC/D) and mDA neuronal (Foxa2, Nurr1, Girk2) markers was faithfully co-localized in virtually all TH+ cells in the grafts generated by VC-treated VM-NSC transplantation (G-K of FIG. 13b). The TH+ mDA neurons in the graft were neighbored by cells positive for dopamine and cAMP-regulated phosphoprotein-32 (DARPP-32), a marker for striatal post-synaptic neurons receiving signal from nigral mDA neurons (L of FIG. 13b). These findings collectively suggest that VC treatment during VM-NSC expansion yielded therapeutically competent donor cells for use in cell therapeutic approaches to treating PD.

### 3. Discussion

The most serious drawback associated with utilizing tissue-specific stem cell cultures in research and therapies is that their original properties and functionalities are altered during in vitro culturing. Cultured cells are expected to be exposed to cellular stresses during in vitro cell expansion and passaging. Since ROS is a major molecule causing loss of cell functionality associated with injury and aging, we tested whether scavenging ROS by anti-oxidant treatment could rescue the loss of DA neurogenic potential which occurs during culture of VM-NSCs. Our data showed that none of the antioxidants tested, except VC, prevented this culture-dependent change. These results suggest that ROS or cellular aging/senescence was not the mechanism responsible for the loss of DA neuron yield in cultured VM-NSCs. By contrast, cellular aging/senescence is a leading molecular mechanism causing loss of functionality in stem cells present in adult tissues, and anti-senescence reagents could prevent culture-dependent changes in stem cell cultures derived from adult tissues. As DA neuron formation occurred in VM tissues at an early embryonic stage, our cultures were derived from embryonic VMs. Based on these findings, it was likely that cellular aging/senescence was not the critical factor responsible for the functionality changes in cultured stem cells derived from embryonic tissues; although it was the major mechanism for loss of stem cell functions derived from adult tissues. Instead, the culture-dependent changes of the NSCs derived from embryonic VM tissues were likely to be associated with the developmental program.

The expression of midbrain factors Foxa2 and Nurr1 is critical for mDA neuron survival, functions and phenotype maintenance. However, the expression of these factors in mDA neurons present in the midbrain is reduced with aging or exposure to a toxin. Due to an immune-inflammatory reaction after cell transplantation, since the brain tissue environment is considered to be hostile to the grafted cells, it seems that the hostile brain environment is the main reason that midbrain-specific factor expression was rarely detected in grafted mDA neurons in the control (C-F of FIG. 13b). Therefore, expression of midbrain-specific factors in grafted mDA neurons is critical for the success of cell therapy for PD. In addition to increased DA neuronal yield, VC pre-treatment of donor NSCs maintained midbrain-marker expression in grafted mDA neurons long after transplantation, which was closely related to enhanced grafted cell survival and functions, and ultimately enhanced therapeutic outcomes. Another characteristic of NSCs altered during in vitro expansion as well as in vivo brain development is transition of NSC differentiation propensity from neuronal to astrocytic differentiation (FIGS. 12d, 12e). Based on the observed effect of VC in preventing culture-dependent changes, it was expected that VC could also prevent neuron-to-astrocyte transition during in vitro culturing. However, contrary to our expectations, VC treatment rather strongly enhanced astrocytic differentiation from cultured VM-NSCs via DNA/histone demethylation in the GFAP promoter region (FIG. 12c), indicating that VC-mediated effects on VM-NSC cultures are not completely associated with prevention of culture- or development-dependent processes. Based on the physiologic neurotrophic actions of brain astrocytes, transplantation of astrocytes alone or together with neurogenic donor cells has become a therapeutic potential for treating intractable brain disorders. Thus, the VC effect on astrocyte differentiation is another beneficial contributor to attain improved therapeutic outcomes achieved by transplanting VC-treated NSCs, in which astrocytes differentiated from the grafted NSCs exert trophic support for neuronal differentiation/maturation and survival in grafted brains. It was noted that TH+DA neuronal cells in the grafts formed by VC-treated NSCs were more abundantly surrounded by GFAP+ astrocytes (FIG. 140.

In the present invention, we demonstrated that VC exerted its observed effects via DNA/histone demethylation-based epigenetic control on DA neuron developmental genes. A similar epigenetic control mechanism was demonstrated in our previous study by treatment with VC during NSC differentiation. [He, X. B., Kim, M., Kim, S. Y., Yi, S. H., Rhee, Y. H., Kim, T., Lee, E. H., Park, C. H., Dixit, S., Harrison, F. E., et al. (2015). Vitamin C facilitates dopamine neuron differentiation in fetal midbrain through TET1- and JMJD3-dependent epigenetic control manner. Stem Cells 33, 1320-1332]. However, epigenetic regulation and gene expression induced by VC treatment of differentiating NSCs was limited to terminally differentiated DA neuron genes such as TH and DAT. Thus, without effects on the expression of midbrain-specific factors and differentiated cell survival, the therapeutic value of the previous VC treatment method seems to be marginal. By contrast, the effect of VC treatment during NSC expansion covered changes in a wide range of mDA neuron developmental and phenotype genes, such as those acting at early undifferentiated stages of VM-NSCs (Foxa2, Lmx1a), intermediate mDA neuron progenitors (Nurr1), and terminally differentiated mDA neurons (TH). Interestingly, after VC induced epigenetic changes by activating Tet and Jmjd enzyme activities in proliferating/undifferentiated NSCs, the epigenetic status of those genes was maintained in differentiated mDA neurons long after VC withdrawal. These findings suggest that transient VC treatment can induce a stable and long-lasting epigenetic change in mDA neuronal genes. It is also possible that VC treatment only triggers the mDA neuron developmental cascade by directly promoting the expression of Foxa2 and Lmx1a, the master regulators expressed in undifferentiated VM-NSCs. Induction of the later developmental factor expressions subsequently follows in the facilitated developmental cascade. The later developmental factors may take over VC-mediated epigenetic regulatory actions and contribute to the maintenance of the epigenetic status during later differentiation stages. The sustained open DNA/chromatin structures surely contributed to the generation of mDA neurons expressing midbrain-specific markers and promoted resistance to toxic stimuli both in vitro and in vivo long after transplantation.

In order to apply VC treatment in the clinical setting of PD cell therapy, the VC effects observed in this study should be replicated in human NSC cultures. Thus, we treated proliferating NSCs derived from human embryonic stem cells (hESCs) [Rhee, Y. H., Ko, J. Y., Chang, M. Y., Yi, S. H., Kim, D., Kim, C. H., Shim, J. W., Jo, A. Y., Kim, B. W., Lee, H., et al. (2011). Protein-based human iPS cells efficiently generate functional dopamine neurons and can treat a rat model of Parkinson disease. The Journal of clinical investigation 121, 2326-2335.] with VC, and differentiation of the human NSCs was induced without VC. Similar to rodent VM-NSC cultures, VC treatment greatly promoted differentiating/differentiated cell survival in hESC-NSC cultures (FIG. 15a). In addition, TH+DA neuronal yields were significantly enhanced in the cultures differentiated from the VC-treated human NSCs (FIG. 15b). Compared to untreated control cultures, TH+ cells in the VC-treated cultures exhibited more extensive neurite branching (FIG. 15c). Along with an increase of TH mRNA expression (FIG. 15d), the levels of 5hmC at human TH promoter regions (two consensus Nurr1 binding sites; NGFI-B response element) were enhanced at the expense of 5mC levels by VC treatment, but H3K9m3/H3K27m3 levels, which significantly decreased in the rat TH promoter after VC-pretreatment, were undetectable in the human TH promoter regions (FIG. 16e), indicating similarity and dissimilarity between rodent and human TH promoters in VC-mediated epigenetic control. The observed benefits of VC treatment in human NSC-DA differentiation and differentiated cell survival are at least applicable in PD cell therapeutic approaches. Considering the normally high VC levels in brain cells, VC treatment of donor cells is the same as a physical environment, and is simple and safe. Therefore, use of this VC pre-treatment strategy during preparation of donor NSCs could become a PD cell therapy option in the clinical setting.

### 4. Conclusion

Cultured neural stem cells (NSCs) are regarded as a potential systematic cell source to treat Parkinson's disease (PD). However, the therapeutic potential of these cultured NSCs is lost during culturing. The inventors confirmed that, the vitamin C (VC) treatment performed during ventral midbrain (VM)-derived NSC expansion and passaging prevents the loss of NSC characteristics associated with therapeutic functions, such as yields of differentiated midbrain-type dopamine (mDA) neurons, the expression of a midbrain-specific factor in differentiated mDA neurons, and the survival of differentiated cells, thereby enabling the expansion of VM-NSCs with an excellent therapeutic potential even after long-term culturing. VC acted by upregulating a series of mDA neuron-specific developmental and phenotype genes via DNA hydroxymethylation/demethylation and repressive histone code (H3K9m3, H3K27m3) demethylation at associated gene promoter regions. Notably, the epigenetic changes induced by transient VC treatment were sustained long after VC withdrawal. Accordingly, transplantation of VC-treated NSCs resulted in improved behavioral restoration, along with enriched DA neuron engraftment, which faithfully expressed midbrain-specific markers in PD model rats. These results indicate that VC treatment to donor NSCs could be a simple, efficient, and safe therapeutic strategy for PD in the future.

The present invention confirmed that the VC treatment performed during VM-NSC expansion (during the culturing of donor cells) is very effective for cell therapy for a neurological disease. The VC effect was achieved by consistently inducing a series of development of mDA neurons and the expression of phenotype-related genes for a long period by an epigenetic control mechanism. Therefore, the transient VC treatment performed during preparation of donor cells caused improvement of neurological disease-associated behaviors after transplantation into neurological disease model rats. This improvement was accompanied by transplantation of abundant DA neurons sufficiently expressing midbrain-specific factors. Based on this finding, VC treatment of donor NSCs is expected to be very useful for cell therapy for a neurological disease.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A cell therapeutic agent, comprising vitamin C-treated ventral midbrain-type neural stem cells (VM-NSCs).

2. The agent according to claim 1, wherein the VM-NSCs express midbrain-specific factors including an NSC-specific factor.

3. The agent according to claim 2, wherein the midbrain-specific factor is selected from the group consisting of Foxa2, Lmx1a and Nurr1.

4. The agent according to claim 1, wherein vitamin C is treated during VM-NSC expansion.

5. A method of preparing ventral midbrain-type neural stem cells (VM-NSCs) which express VM-specific factors, comprising:
treating vitamin C during VM-NSC expansion.

6. The method according to claim 5, wherein the vitamin C is treated at 50 to 300 μM.

7. A method of treating a neurological disease, comprising:
administering vitamin C-treated ventral midbrain-type neural stem cells (VM-NSCs) to a subject.

8. The method according to claim 7, wherein the neurological disease is selected from the group consisting of Parkinson's disease, Alzheimer's disease, Huntington's disease, amyotrophic lateral sclerosis, stroke, ischemia and neuronal diseases caused by spinal cord injury.


SEQUENCE LISTING (55 sequences)

SEQ ID NO: 1 - DNA, 12 bp, Artificial Sequence
  Prediction Binding Sites
gaaagttgattt

SEQ ID NO: 2 - DNA, 12 bp, Artificial Sequence
  Prediction Binding Sites
ctctattgtttt

SEQ ID NO: 3 - DNA, 12 bp, Artificial Sequence
  Prediction Binding Sites
tattatttatat

SEQ ID NO: 4 - DNA, 12 bp, Artificial Sequence
  Prediction Binding Sites
taatatttcctt

SEQ ID NO: 5 - DNA, 10 bp, Artificial Sequence
  Prediction Binding Sites
ttccgagaag

SEQ ID NO: 6 - DNA, 20 bp, Artificial Sequence
  Gapdh Forward primer
ctcatgaccacagtccatgc

SEQ ID NO: 7 - DNA, 20 bp, Artificial Sequence
  Gapdh Reverse primer
ttcagctctgggatgacctt

SEQ ID NO: 8 - DNA, 21 bp, artificial sequence
  Th Forward primer
aagggcctctatgctacccaa

SEQ ID NO: 9 - DNA, 20 bp, Artificial Sequence
  Th Reverse primer
tgcattgaaacacgcggaag

SEQ ID NO: 10 - DNA, 24 bp, Artificial Sequence
  Dat Forward primer
ggaccaatgttcttcagtggtggc

SEQ ID NO: 11 - DNA, 20 bp, Artificial Sequence
  Dat Reverse primer
gggccggcgaggggcttgac

SEQ ID NO: 12 - DNA, 20 bp, Artificial Sequence
  Vmat2 Forward primer
ggcttccttctcaactcccc

SEQ ID NO: 13 - DNA, 20 bp, Artificial Sequence
  Vmat2 Reverse primer
ggctctgacgtcacggatag

SEQ ID NO: 14 - DNA, 22 bp, Artificial Sequence
  Aadc Forward primer
tggcaatttaaaggctctggac

SEQ ID NO: 15 - DNA, 21 bp, Artificial Sequence
  Aadc Reverse primer
cagaaaaaggctgtcctgggg

SEQ ID NO: 16 - DNA, 20 bp, Artificial Sequence
  Foxa2 Forward primer
tatgtgggcgctggaatgag

SEQ ID NO: 17 - DNA, 21 bp, Artificial Sequence
  Foxa2 Reverse primer
ggcaccttgagaaagtcgttg

SEQ ID NO: 18 - DNA, 20 bp, Artificial Sequence
  Lmx1a Forward primer
tatacaacgttgcccacccc

SEQ ID NO: 19 - DNA, 20 bp, Artificial Sequence
  Lmx1a Reverse primer
gatggggtttcccactctgg

SEQ ID NO: 20 - DNA, 20 bp, Artificial Sequence
  Nurr1 Forward primer
cggtttcagaagtgcctagc

SEQ ID NO: 21 - DNA, 20 bp, Artificial Sequence
  Nurr1 Reverse primer
ttgcctggaacctggaatag

SEQ ID NO: 22 - DNA, 21 bp, Artificial Sequence
  Human U6 snRNA Forward primer
tcgcttcggcagcacatatac

SEQ ID NO: 23 - DNA, 20 bp, Artificial Sequence
  Human U6 snRNA Reverse primer
tgcgtgtcatccttgcgcag

SEQ ID NO: 24 - DNA, 21 bp, Artificial Sequence
  Human Th Forward primer
gagtacaccgccgaggagatt

SEQ ID NO: 25 - DNA, 22 bp, Artificial Sequence
  Human Th Reverse primer
gcggatatactgggtgcactgg

SEQ ID NO: 26 - DNA, 20 bp, Artificial Sequence
  Foxa2 Promoter I Forward primer
ccacctactgccctgtttgt

SEQ ID NO: 27 - DNA, 20 bp, Artificial Sequence
  Foxa2 Promoter I Reverse primer
ctcaggcctttttctggcta

SEQ ID NO: 28 - DNA, 20 bp, Artificial Sequence
  Foxa2 Promoter II Forward primer
tctctgggttccctgtgttc

SEQ ID NO: 29 - DNA, 20 bp, Artificial Sequence
  Foxa2 Promoter II Reverse primer
cacttgggtctgcatccttt

SEQ ID NO: 30 - DNA, 20 bp, Artificial Sequence
  Foxa2 Promoter III Forward primer
agagggacgggggaatagac

SEQ ID NO: 31 - DNA, 20 bp, Artificial Sequence
  Foxa2 Promoter III Reverse primer
gtactggactcccgagatgc

SEQ ID NO: 32 - DNA, 19 bp, Artificial Sequence
  Lmx1a Promoter FB Forward primer
agctcgcccatcaggtaag

SEQ ID NO: 33 - DNA, 20 bp, Artificial Sequence
  Lmx1a Promoter FB Reverse primer
gaacgtctgcaggcgcaaag

SEQ ID NO: 34 - DNA, 20 bp, Artificial Sequence
  Nurr1 Promoter FB1 Forward primer
ccaccctcgcacccaaatta

SEQ ID NO: 35 - DNA, 20 bp, Artificial Sequence
  Nurr1 Promoter FB1 Reverse primer
aaacacagcatcaaagccgc

SEQ ID NO: 36 - DNA, 22 bp, Artificial Sequence
  Nurr1 Promoter FB2 Forward primer
agcattgtggagaaggtgctaa

SEQ ID NO: 37 - DNA, 20 bp, Artificial Sequence
  Nurr1 Promoter FB2 Reverse primer
gggcaagtgaattggtgttc

SEQ ID NO: 38 - DNA, 20 bp, Artificial Sequence
  Th Promoter NB1 Forward primer
agaggatgcgcaggaggtag

SEQ ID NO: 39 - DNA, 20 bp, Artificial Sequence
  Th Promoter NB1 Reverse primer
gtcccgagttctgtctccac

SEQ ID NO: 40 - DNA, 20 bp, Artificial Sequence
  Th Promoter NB2 Forward primer
tcctggaggggactttatga

SEQ ID NO: 41 - DNA, 20 bp, Artificial Sequence
  Th Promoter NB2 Reverse primer
ctggatttcctaagggctca

SEQ ID NO: 42 - DNA, 20 bp, Artificial Sequence
  Th Promoter NB3 Forward primer
gggtgtggatgctaactgga

SEQ ID NO: 43 - DNA, 20 bp, Artificial Sequence
  Th Promoter NB3 Reverse primer
agtggtagccccattctcag

SEQ ID NO: 44 - DNA, 20 bp, Artificial Sequence
  Th Promoter 10CpG Forward primer
tctcagagcaggatgccaac

SEQ ID NO: 45 - DNA, 20 bp, Artificial Sequence
  Th Promoter 10CpG Reverse primer
acaagatgggaccaagaacc

SEQ ID NO: 46 - DNA, 21 bp, Artificial Sequence
  Gfap Promoter STAT3 Binding Forward primer
tgactcaccttggcatagaca

SEQ ID NO: 47 - DNA, 19 bp, Artificial Sequence
  Gfap Promoter STAT3 Binding Reverse primer
cagtgaggcatacggcaag

SEQ ID NO: 48 - DNA, 20 bp, Artificial Sequence
  Human Th Promoter NRBE1 Forward primer
gggttgtcctcaagggagtt

SEQ ID NO: 49 - DNA, 20 bp, Artificial Sequence
  Human Th Promoter NRBE1 Reverse primer
ctatggcccactgctagctc

SEQ ID NO: 50 - DNA, 20 bp, Artificial Sequence
  Human Th Promoter NRBE2 Forward primer
tccctttgctttgactgagc

SEQ ID NO: 51 - DNA, 20 bp, Artificial Sequence
  Human Th Promoter NRBE2 Reverse primer
gacttctcgaaggcctaccc

SEQ ID NO: 52 - DNA, 20 bp, Artificial Sequence
  Tph2  Promoter Forward  primer
caaagggctactcgacctat

SEQ ID NO: 53 - DNA, 20 bp, Artificial Sequence
  Tph2 Promoter Reverse primer
gtgctgaagagcagtgcgct

SEQ ID NO: 54 - DNA, 20 bp, Artificial Sequence
  Gad67 Promoter Forward primer
agacacctgcaaaggagccc

SEQ ID NO: 55 - DNA, 20 bp, Artificial Sequence
  Gad67 Promoter Reverse primer
acagggagctggaccctccc